(19) United States
(12) Patent Application Publication
(10) Pub. No.: US 2003/0105043 A1
(43) Pub. Date: Jun. 5, 2003

(54) Apoptosis-inducing ribozymes

(76) Inventors: Shuk-Mei Ho, Lexington, MA (US); Kin-Mang Lau, Worcester, MA (US); Kai-Fai Lee, Hong Kong (HK)

Correspondence Address: FISH & RICHARDSON PC, 225 FRANKLIN ST, BOSTON, MA, 02110, US

(21) Appl. No.: 10/033,024

(22) Filed: Oct. 31, 2001

Related U.S. Application Data
(60) Provisional application No. 60/244,709, filed on Oct. 31, 2000.

(51) Int. Cl. A61K 48/00; C12N 15/85

(52) U.S. Cl. 514/44; 435/455; 435/320.1

(57) ABSTRACT

Ribozymes directed against metallothionein mRNAs have been designed and tested in vitro and in living cells. Induction of apoptosis in cultured human cancer cells was demonstrated using such ribozymes. Based on these developments, the invention features new ribozymes and methods of inducing apoptosis in a human cancer cell in vitro or in vivo. The method includes introducing into the cell a ribozyme that inhibits metallothionein expression.

### CROSS-REFERENCE TO RELATED APPLICATIONS

This application claims priority to provisional U.S. Application Serial No. 60/244,709, filed Oct. 31, 2000, which is incorporated herein by reference in its entirety.

### FEDERALLY SPONSORED RESEARCH

Work on this invention was supported by NIH Grants, CA15776 and CA62269. Therefore, the federal government has certain rights in the invention.

### TECHNICAL FIELD

This invention relates to molecular biology, cell biology, oncology and urology.

### BACKGROUND

Metallothioneins (MTs) are found in most tissues of a wide variety of vertebrate and invertebrate animals, and in plants (Kagi et al., 1988, Biochemistry 27:8509-8515; Vallee, Neurochem Int. 27:23-33; Bremmer, 1991, Methods Enzymol. 205:25-35). Metallothioneins are characterized by low molecular weight (about 60 amino acids), high cysteine content (25-30%), lack of aromatic amino acid residues, and affinity for heavy metal ions, including zinc, copper, mercury, and cadmium. Although the precise biological functions of metallothioneins are not fully understood, it has been suggested that metallothioneins are involved in detoxification of heavy metals, homeostasis trafficking of essential trace elements such as zinc and copper, scavenging of free radicals, and protection against alkylating agents (Nath et al., 1988, CRC Crit. Rev. Food Sci. Nutr. 27:41-85; Lohrer et al., 1989, Carcinogenesis 10:2279-2284; Templeton et al., 1991, Methods Enzymol. 205:11-24; Ebadi et al., 1995, Neurochem. Int. 29:159-166). Also, it has been suggested that they may be involved in resistance to anti-cancer drugs by at least some types of cancer cells (Kondo et al., 1995, Cancer Res. 55:474-477; Satoh et al., Cancer Res. 54:5255-5257).

Ribozymes are small catalytic RNA molecules. Types of ribozymes that occur naturally in genomes of RNA viruses and in virus-related RNAs include self-splicing Group I and Group II introns, precursor tRNA-processing RNase P, hammerhead ribozymes, hairpin ribozymes, and delta virus ribozymes. Among these, the hammerhead ribozymes are well characterized and most widely used in experimental studies. A hammerhead ribozyme includes a central catalytic core flanked by two antisense side arms. The side arms hybridize with the substrate RNA, using Watson-Crick base pairing, thereby providing sequence specificity to the endonuclease action of the catalytic core. The catalytic action involves cleavage of a specific phosphodiester bond on the targeted RNA substrate. The cleavage occurs at the 3′ end of a cleavage triplet, 5′-NUH-3′ on the RNA substrate, where N is any nucleotide and H is adenine (A), uracil (U), or cytosine (C). Guanine (G) and cytosine (C) are the preferred first and third bases of the triplet.

### SUMMARY

Ribozymes directed against metallothionein mRNAs have been designed and tested in vitro and in living cells. In tests with cultured human cancer cells, induction of apoptosis has been demonstrated using such ribozymes.

Based on these developments, the invention features a method of inducing apoptosis in a human cancer cell in vitro or in vivo. The method includes introducing into the cell a ribozyme that inhibits metallothionein expression. In some embodiments, introducing the ribozyme into the cell includes injecting the ribozyme directly into a tumor comprising the cell in vivo. Preferably, the ribozyme is a hammerhead ribozyme. Useful ribozymes have or include one of the following nucleotide sequences:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

The human cancer cell can be, for example, a prostate cancer cell, a breast cancer cell, or an ovarian cancer cell.

The invention also features a method of inducing apoptosis in a human cancer cell by introducing into the cell a nucleic acid vector containing a nucleotide sequence encoding a ribozyme that inhibits metallothionein expression. The nucleotide sequence can be operatively linked to a tissue-specific promoter suitable for the type of cancer being treated. For example, the ribozyme-encoding nucleotide sequence can be operatively linked to a prostate tissue-specific promoter, a breast tissue-specific promoter, or an ovarian tissue-specific promoter.

The invention also features a method of inhibiting growth of a tumor. The method includes introducing into cells of the tumor a ribozyme that inhibits metallothionein expression. The ribozyme can be introduced into the cells of the tumor by injecting the ribozyme directly into the tumor. The invention also features a method of inhibiting growth of a tumor, wherein a nucleic acid vector containing a nucleotide sequence encoding a ribozyme that inhibits metallothionein expression is introduced into cells of the tumor.

The invention also features a method of enhancing the effectiveness of chemotherapy against cancer cells. The method includes introducing into the cancer cells of a patient a ribozyme that inhibits metallothionein expression; and administering to the patient a therapeutically effective amount of a chemotherapy agent. Examples of chemotherapy agents that can be used in this ribozyme/chemotherapy combination treatment include cisplatin, estramustine, vinblastine, etopside and other topoisomerase II inhibitors, Paclitaxel, taxotere, docetaxel, doxorubicin, ketocanazole, and cyclophosphamide. The chemotherapy agent can be administered according to conventional medical practice, and the ribozyme or vector for expressing the ribozyme can be injected directly into an in vivo tumor comprising the cells.

The invention also features a method of enhancing the effectiveness of radiation therapy against cancer cells. The method includes introducing into the cancer cells of a patient a ribozyme that inhibits metallothionein expression and administering to the patient a therapeutically effective amount of radiation therapy. Examples of types of radiation therapy that can be used in this ribozyme/radiation therapy combination treatment include external beam radiation, e.g., for prostate cancer; brachytherapy, e.g., for thyroid cancer; ¹²⁵I administration, e.g., for ovarian cancer; and 125I estrogen.

The invention also features a ribozyme that has or includes one of the following nucleotide sequences:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

The invention also features a nucleic acid vector containing sequences encoding one or more of SEQ ID NO: 1, SEQ ID NO: 2, SEQ ID NO: 3, SEQ ID NO: 4, SEQ ID NO: 5, SEQ ID NO: 6, and SEQ ID NO: 7, wherein the ribozyme coding sequence(s) is (are) operatively linked to one or more expression control sequences. In some embodiments, the vector contains nucleotide sequences encoding ribozymes Hu MT-Ia and Hu MT-Ie/r; ribozymes Hu MT-If and Hu MT-Ie/r; or ribozymes Hu MT-Ib and Hu MT-Ighlx/-II.

Unless otherwise defined, all technical and scientific terms used herein have the same meaning as commonly understood by one of ordinary skill in the art to which this invention belongs. In case of conflict, the present application, including definitions, will control. All publications, patents and other references mentioned herein are incorporated by reference.

Although methods and materials similar or equivalent to those described herein can be used in the practice or testing of the present invention, the preferred methods and materials are described below. The materials, methods and examples are illustrative only and not intended to be limiting. Other features and advantages of the invention will be apparent from the detailed description and from the claims.

### DESCRIPTION OF DRAWINGS

FIG. 1 is a representation of double-stranded DNA sequences used in the construction of plasmids for the production of active and inactive ribozymes targeting metallothionein transcripts (SEQ ID NOS: 37-44). Two pairs of overlapping synthetic oligonucleotides, representing forward and reverse primers (boxed), were used to generate fragments encoding the active ribozymes, Rz1-2 and Rz4-9 by PCR. RzI-F, the forward primer, and RzI-R, the reverse primer, were used to generate Rz1-2, which was designed to cleave mouse and rat MT-I mRNA. RzII-F and RzII-R were the forward and reverse primers, respectively, used in the synthesis of Rz4-9, which was designed to cleave MT-II mRNA. Two enzymatically inactive ribozymes, Rz-2-6 and Rz3-3, were obtained by substituting the forward primer for Rz1-2 synthesis and the reverse primer for Rz4-9 production with a mutant oligonucleotide, RzI-M, in the PCR. RzI-M derives from the RzI-F sequence with a two-base substitution (boxed). Asymmetrical restriction sites were created at the ends of these constructs for directional cloning into the pClneo expression vector.

FIGS. 2A and 2B are schematic representations of the sequence structures of Rz1 -2 (SEQ ID NO: 46) and Rz4-9 (SEQ ID NO: 48) (respectively) and their predicted interaction with rat MT-I RNA (SEQ ID NO: 45) and MT-II RNA (SEQ ID NO: 47), respectively. Each ribozyme consists of a ribozyme core sequence, which harbors the catalytic activity and two annealing arms (Stem I and Stem III). The catalytic core is made up of conserved sequences, CUGAUGA on the 5′ side of Stem II and GAAA on the 3′ side of Stem II. The three GC pairs (Stem II) and the GUGA tetra loop in the ribozyme core sequence stabilize this structure. The substrate (MT mRNA) and the Rz hybridize to form Stem I and Stem III. Sequence complementation between strands in Stem I and Stem III confers substrate specificity. The cleavage site (arrowed) is located on the 3′ side of the cleavage triplet sequence (italicized) GUC in rat MT-I mRNA and AUC in MT-II mRNA and demonstrated in the primer extension analyses.

FIG. 3 is a table showing the sequence comparison between rodent and human metallothionein mRNAs in the regions recognized by ribozymes. The cleavage triplets are also shown. PM indicates perfect match. An asterisk indicates mismatch predicted to affect the cleavage step based on the finding of a study by Werner et al. (1995, Nucleic Acids Res. 23:2092-2096). The expected effectiveness activities of two ribozymes on different isoforms of rodent and human metallothionein mRNAs are predicted based on the findings of Werner et al. (supra).

FIG. 4 is a graph summarizing data on enzymatic kinetics of ribozyme on rat metallothionein cRNAs. The observed rate (k), is described herein. Concentration of ribozyme is a representation of the data from autoradiograms. The slopes represent k_{cat}/Km values of two Rzs. Filled circle, Rz1-2; Open circle, Rz4-9.

FIG. 5 is a graph summarizing data on cadmium (Cd)-induced cytotoxicity in NbE-1 (a prostatic epithelial cell line derived from Noble rats) and clonal lines of NbE-1 containing stably transfected DNA constructs encoding Rz1-2/4-9 (designated NbE-1 (Rz1), NbE-1(Rz2), and NbE-1 (Rz3)). Cells (5×10³ cells per well) were plated on a 96 wells-plate. After 24 hrs for cell attachment, they were exposed to Cd at 0-400 μM for 4 days. Cell viability was determined by the MTT Cell Viability Assay kit (Boehringer-Mannheim, Indianapolis, Ind.) at the end of treatment. Data are represented by two individual experiments in 4-6 replicates.

FIG. 6 is a representation of complete, double-stranded sequences for DNA constructs encoding ribozymes targeting human metallothionein transcripts. Boxes indicate two pairs of overlapping synthetic oligonucleotides, representing forward and reverse primers that can be used to generate fragments encoding the ribozymes designated Hu MT-Ia (SEQ ID NOS: 49-50), Hu MT-Ie/r (SEQ ID NOS: 51-52), Hu MT-If (SEQ ID NOS: 53-54), Hu MT-Ib (SEQ ID NOS: 55-56), and Hu MT-Ighlx/-II (SEQ ID NOS: 57-58). Restriction endonuclease sites useful in cloning and vector construction are indicated.

FIGS. 7A-7E are schematic representations of the sequence structures of human ribozymes Hu MT-Ia (SEQ ID NO: 60), Hu MT-Ie/r (SEQ ID NO: 68), Hu MT-If (SEQ ID NO: 66), Hu MT-Ib (SEQ ID NO: 62), and Hu MT-Ighlx/-II (SEQ ID NO: 64) and their predicted interaction with human metallothionein mRNA targets (SEQ ID NOS: 59, 61, 63, 65, and 67, respectively). Each ribozyme includes a ribozyme core sequence, which harbors the catalytic activity and two annealing arms (Stem I and Stem III). The upper sequence in each FIG. is part of the targeted mRNA. FIG. 7A shows ribozyme Hu MT-IaRz interacting with human MT-Ia RNA. FIG. 7B shows ribozyme Hu MT-IbRz interacting with human MT-Ib RNA. FIG. 7C shows ribozyme Hu MT-Ighlx/-IIRz interacting with human MT-Ig, Ih, Il, Ix, and MT-II RNA. FIG. 7D shows ribozyme Hu MT-If Rz interacting with human MT-If RNA. FIG. 7E shows ribozyme Hu MT-Ie/r interacting with human MT-Ie/r RNA.

FIGS. 8A-8B are graphs from experiments on induction of cell death in PC-3 cells by ribozymes targeted to metallothionein. FIG. 8A is a bar graph showing the results of experiments in which three different concentrations of Rz-4-9 (0.5 μg, 1 μg and 2 μg) were transfected into PC-3 cells and the amount of cell death determined after 48 hours. pCl-neo vector (empty vector) and Mutant (pCl-neo containing an enzymatically inactive mutant ribozyme) are controls. Asterisk (*) represents a statistically significant change. FIG. 8B is a set of histograms of cell number and DNA content in PC-3 cells treated with various concentrations of Rz-4-9, empty vector (Vector) and an enzymatically inactive mutant (Mutant). Data represent an average of four experiments.

FIGS. 9A-9B are graphs from experiments on induction of cell death in SKOV-3 cells by ribozymes targeted to metallothionein. FIG. 9A is a bar graph showing the results of experiments in which three different concentrations of Rz-4-9 (0.5 μg, 1 μg and 2 μg) were transfected into SKOV-3 cells and the amount of cell death determined after 48 hours. pCl-neo vector (empty vector) and Mutant (an enzymatically inactive mutant ribozyme) are controls. Asterisk (*) represents a statistically significant change. FIG. 3B is a set of histograms of cell number and DNA content in SKOV-3 cells treated with various concentrations of Rz-4-9, empty vector (Vector) and an enzymatically inactive mutant (Mutant). Data represent an average of four experiments.

FIGS. 10A-10B are graphic results of reverse transcriptase (RT) PCR analyses of transcript levels in PC-3 cells treated with vectors encoding ribozyme Rz4-9 and controls. FIG. 10 is a fluorogram of amplimers of MT-IIa, MT-If, bc1-2, c-myc, and 18S rRNA cDNA obtained from cells treated with various concentrations of Rz 4-9, empty vector (control) and mutant ribozyme (control). FIG. 10B Panel B represents a quantitative analysis of gene expression in the form of histograms. The values represent a mean of four experiments. The bands were quantitated in ImageQuant™ (Molecular Dynamics, Sunnyvale, CA) and plotted after normalizing the intensity of the bands with respect to the 18S rRNA controls. Asterisk (*) represents a statistically significant change

FIGS. 11A-11B are graphic results of reverse transcriptase (RT) PCR analyses of levels of mRNAs in SKOV-3 cells treated with vectors encoding ribozymes and controls. FIG. 11A is a fluorogram of amplimers of MT-IIa, MT-If, bc1-2, c-myc, and 18S rRNA cDNA obtained from cells treated with various concentrations of Rz 4-9, empty vector (control) and mutant ribozyme (control). FIG. 11B represents a quantitative analysis of expression in the form of histograms. The values represent a mean of four experiments. The bands were quantitated in ImageQuant and plotted after normalizing the intensity of the bands with respect to the 18S rRNA controls. Asterisk (*) represents a statistically significant change

FIGS. 12A-12E are plots of Annexin V and propidium iodide (PD stained PC-3 cells transfected with various concentrations of Rz 4-9 and controls analyzed by FACS. FIG. 12A: PC-3 cells transfected with pCl-neo (empty) vector, FIG. 12B: PC-3 cells transfected with 0.5 μg Rz 4-9, FIG. 12C: PC-3 cells transfected with 1 μg Rz 4-9, FIG. 12D: PC-3 cells transfected with 2 μg Rz 4-9, FIG. 12E: PC-3 cells transfected with enzymatically inactive ribozyme. The lower left quadrangle of each figure represents live viable cells, the lower right quadrangle represents early apoptotic cells, and the upper right quadrangle represents late apoptotic or necrotic cells. The upper left quadrangle represents necrotic cells. The percentage of cells in each population is also represented in the insets.

FIGS. 13A-13E are plots of Annexin V and propidium iodide (PI) stained SKOV-3 cells transfected with various concentrations of Rz 4-9 and controls analyzed by FACS. FIG. 13A: SKOV-3 cells transfected with pCl-neo (empty) vector, FIG. 13B: SKOV-3 cells transfected with 0.5 μg Rz 4-9, FIG. 13C: SKOV-3 cells transfected with 1 μg. Rz 4-9, FIG. 13D: SKOV-3 cells transfected with 2 μg Rz 4-9, FIG. 13E: SKOV-3 cells transfected with enzymatically inactive ribozyrne. The lower left quadrangle of each figure represents live viable cells, the lower right quadrangle represents early apoptotic cells, and the upper right quadrangle represents late apoptotic or necrotic cells. The upper left quadrangle represents necrotic cells. The percentage of cells in each population is also represented in the insets.

FIG. 14 is a schematic representation of nucleotide sequences of primers that can be used to generate ribozymes of the invention.

### DETAILED DESCRIPTION

By interfering with metallothionein gene expression, ribozymes used in methods according to the invention induce apoptosis in targeted cancer cells. In addition, the ribozymes render targeted cancer cells more susceptible to radiation, reactive oxygen species and chemotherapeutic agents. Thus, methods of the invention can be employed alone or in combination with conventional radiation therapy or chemotherapy. Ribozymes targeted to mRNAs representing one or more of the 17 human metallothionein genes can be used to treat various human cancers, including prostate cancer, breast cancer, and ovarian cancer.

The ribozyme-based methods of the invention offer advantages over conventional antisense-based methods of limiting metallothionein production in target cells. For example, the ribozymes destroy metallothionein-encoding mRNAs, rather than merely hybridizing with them. Ribozymes act like enzymes and each molecule can be “recycled” to degrade multiple mRNA molecules. A further advantage is that a ribozyme need not have perfect complementarity with a target mRNA in order to destroy the RNA. Hybridization need only be sufficient to enable the catalytic site of the ribozyme to cleave the target mRNA. Therefore, a single ribozyme can be designed to destroy several related mRNAs that encode different metallothioneins more readily than a conventional antisense molecule can be designed to be effective against various mRNAs

Ribozymes

Therapeutic methods according to the invention involve the use of a ribozyme, i.e., an antisense nucleic acid that is directed against a metallothionein RNA and includes a catalytic nucleotide sequence that cleaves the targeted metallothionein RNA. Ribozymes, and their construction and use, are known in the art. See, e.g., Norris et al., 2000, “Design and testing of ribozymes for cancer gene therapy,” Adv. Exp Med. Biol. 465:293-301. See also, Cech, U.S. Pat. No. 5,093,246; Cech, U.S. Pat. No. 5,116,742; Haselhoff et al., 1988, Nature 334:585-591. Employing known nucleotide sequences of metallothionein mRNA molecules together with known properties of ribozymes in general, numerous different ribozymes or ribozyme-encoding vectors useful in the invention can be designed and produced.

In some embodiments of the invention, the ribozyme employed is based on a human metallothionein nucleotide sequence. For useful information on nucleotide sequences encoding human metallothioneins, see, e.g., Karin et al., 1982, “Human metallothionein genes: molecular cloning and sequence analysis of the mRNA,” Nucleic Acids Res. 10:3165-3173; Lambert et al., 1996, “Cloning and sequencing a novel metallothionein I isoform expressed in human reticulocytes,” FEBS Lett. 389:210-212. In other embodiments, however, ribozymes designed on the basis of non-human metallothionein RNAs, e.g., rodent RNAs, are employed. Metallothionein sequences that are useful for designing ribozymes and as targets for ribozymes of the invention include mouse MT-I (Genbank Accession No. S62785), rat MT-I (Genbank Accession No. M11794), human MT-Ib (Genbank Accession No. M13485), human MT-Ie (Genbank Accession No. M10942), human MT-If (Genbank Accession No. M10943), human MT-Ir (Genbank Accession No. X97261), mouse MT-II (Genbank Accession No. K02236), rat MT-II (Genbank Accession No. M11794), human MT-II (Genbank Accession No. M26637), human MT-Ia (Genbank Accession No. K0 1383), human MT-Ie Genbank Accession No. M10942), human MT-I g (Genbank Accession No. J03910), human MT-Ih (Genbank Accession No. X64834), human MT-Il (Genbank Accession No. X76717), human MT-Ir (Genbank Accession No. X97261), and human MT-Ix (Genbank Accession No. X65607).

Specific examples of useful ribozymes based on non-human metallothionein RNAs are two hammerhead ribozymes designated Rz1-2 and Rz4-9. The designs of Rz1-2 and Rz4-9 were based on the 3′ coding regions of rat metallothionein I mRNA and rat metallothionein II mRNA, respectively. The design, generation, and testing of Rz1-2 and Rz4-9 are described in Lee et al., 1999, Toxicol. Appl. Pharmacol. 161:294-301. Based on comparisons of human and rat metallothionein sequences, Rz1-2 is predicted to cleave human MT-1b, and Rz4-9 is predicted to cleave human MT-IIa, MT-Ig, Ih, II, and Ix.

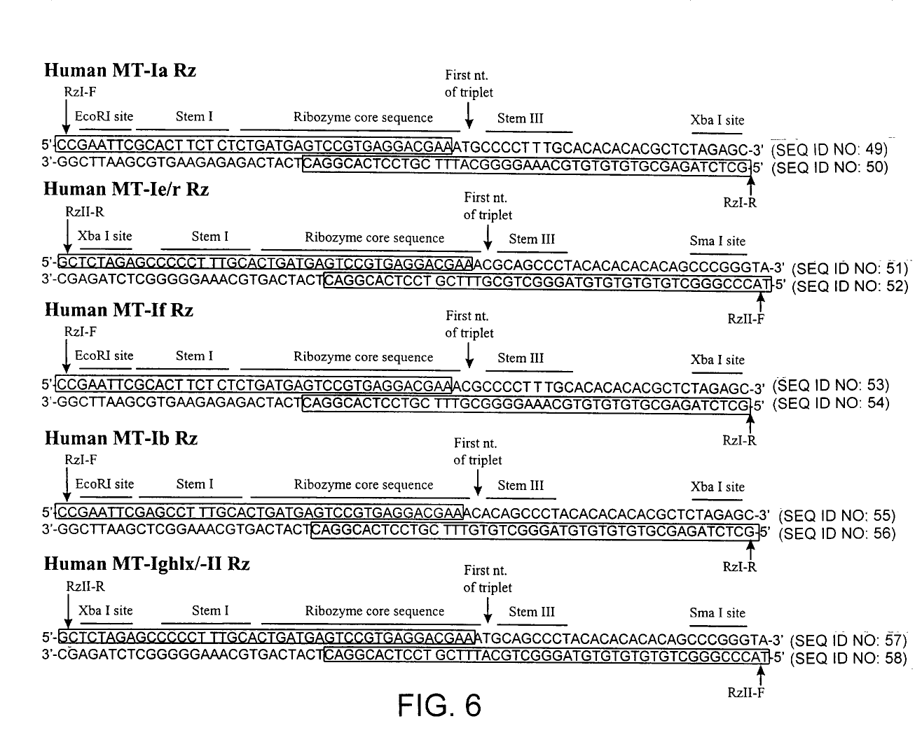

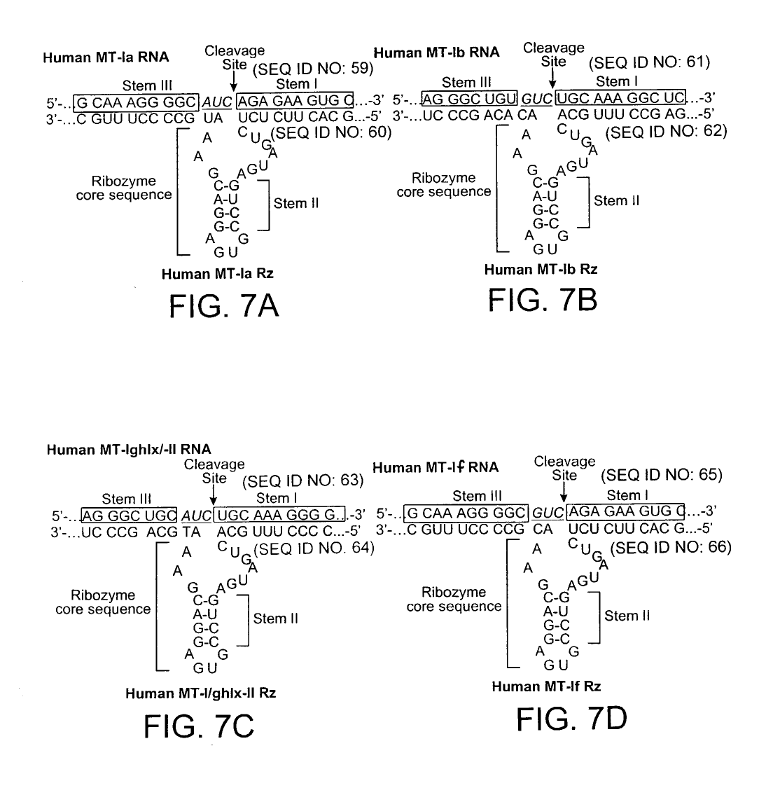

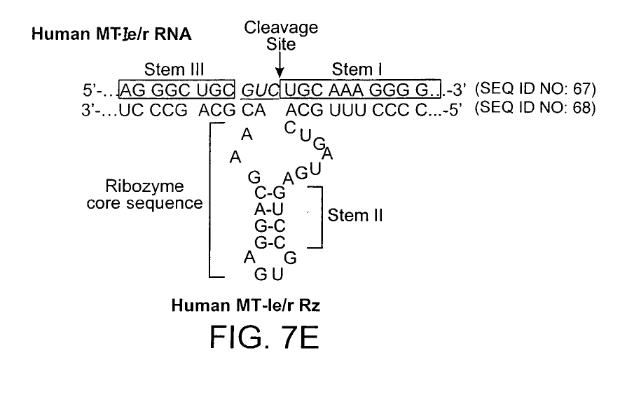

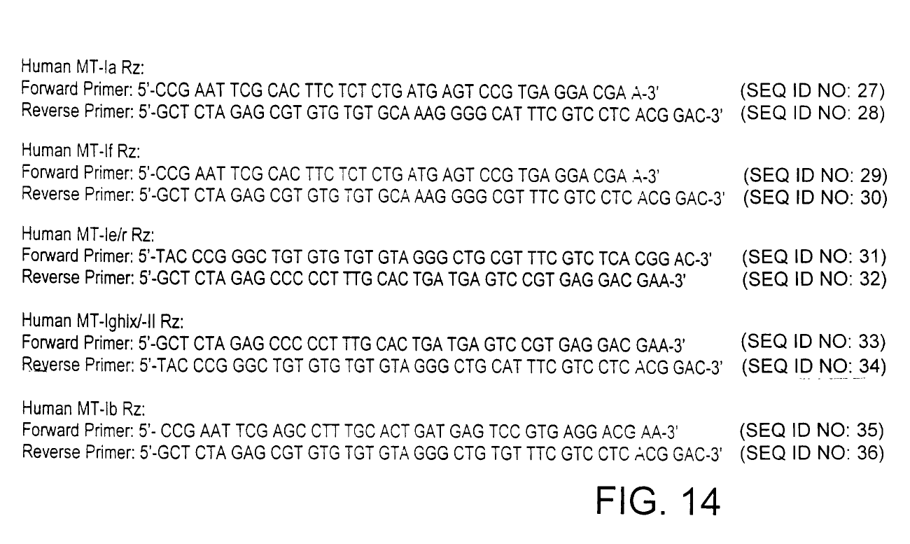

Specific examples of useful ribozymes based on human metallothionein RNAs are hammerhead ribozymes designated Hu MT-Ia, Hu MT-Ie/r, Hu MT-If, Hu MT-Ib, and Hu MT-Ighlx/-II (FIGS. 6 and 7). Each of these ribozymes is predicted to cleave the RNA(s) encoding the human metallothionein(s) indicated in the ribozyme nomenclature. For example, ribozyme Hu MT-Ia is predicted to cleave the mRNA encoding human metallothionein Ia; and ribozyme Hu MT-Ighlx/-II is predicted to cleave the mRNA encoding human metallothionein Ig, Ih, II, Ix and II. DNAs useful for generating these five human ribozymes, including PCR primers (in FIG. 6 and in FIG. 14) and restriction endonuclease sites for cloning, are shown in FIG. 6. The structural relationships between these five human ribozymes and their target mRNAs are shown in FIG. 7. Production of these ribozymes is within ordinary skill in the art. For example, each of these human ribozymes can be produced by substituting the sequences in FIG. 6 in the methods described in Lee et al., 1999 (supra).

In some embodiments of the invention, pre-formed ribozyme molecules are introduced into targeted human cancer cells. Hammerhead ribozymes used in the invention preferably contain from 35 to 55 nucleotides, more preferably from 40 to 50 nucleotides, e.g., approximately 45 nucleotides. Polynucleotides or oligonucleotides in this size range are sufficiently small to be introduced into living cells using known techniques, e.g., techniques employed to introduce conventional antisense oligonucleotides into cultured cells or cells in vivo. Guidance concerning introduction of ribozymes into living cells is provided herein. For additional guidance, see, e.g., Perlman et al., 2000, Cardiovasc. Res. 45:570-578.

Expression Vectors

In some embodiments of the invention, a ribozyme is introduced into living cells indirectly, i.e., by introducing into the cells an expression vector that contains a nucleotide sequence encoding the ribozyme. The vector can contain a single ribozyme coding sequence. Alternatively, it can contain 2 or more, e.g., 3, 4, 5, or 6, ribozyme coding sequences in tandem. Where 2 or more ribozyme coding sequences are present in the vector, the coding sequences can be the same or different. For example, sequence encoding a ribozyme directed against human MT-Ia mRNA can be expressed along with a sequence encoding a ribozyme directed against human MT-Ib mRNA. FIG. 14 shows primer sequences useful for making gene constructs for expression of human ribozymes.

In general, the ribozyme-encoding sequence in the vector is operatively linked to suitable expression control sequences so that a therapeutically effective amount of the ribozyme is expressed in the target cells, i.e., cells into which the vector is introduced. Tissue specific expression of a ribozyme-encoding vector can be achieved by employing a tissue-specific promoter, tissue-specific enhancer element, or both, to drive expression of the ribozyme coding sequence. Tissue specific promoters and enhancer elements useful in the invention are known in the art.

An example of a suitable promoter for driving prostate tissue-specific expression is the prostate specific antigen (PSA) promoter used in combination with a PSA enhancer element. Detailed information regarding the use of a PSA promoter with PSA enhancer, including adenoviral vector construction, for high-level, prostate tissue-specific gene expression in gene therapy for prostate cancer is found in Latham et al., 2000, Cancer Res. 60:334-341. See also, Henderson et al., U.S. Pat. No. 6,057,299. Additional examples of prostate tissue-specific promoters useful in the invention include the mouse mammary tumor virus (MMTV) long terminal repeat (LTR) promoter (Otten et al., 1988, Mol. Endocrinol. 2:143-147) and the probasin (PB) promoter (Matuo et al., 1989, In Vitro Cell Dev. Biol. 25:581-584).

A 12.3 kb fragment upstream of the human kallekrein 2 gene contains an enhancer with androgen response element (ARE) that is selectively active in prostate cancer cells. This human kallekrein 2 enhancer and ARE can be used to achieve prostate tissue-specific expression of ribozymes such as Rz1-2 or Rz4-9, or their human homologs, in methods according to the invention. Details concerning the use of the human kallekrein 2 enhancer and ARE in the construction an adenoviral vector for prostate cancer therapy are described in Yu et al., 1999, Cancer Res. 59:1498-1504. See also, Steiner et al., 1999, Cancer Gene Ther. 6:456-464.

Examples of suitable promoters for driving human breast tissue-specific expression include Muc-1, CEA, PSA, HER-2, Myc, L-plastin and secretory leukoproteinase inhibitor promoters. All of these promoters display differential upregulation in breast cancer. For guidance concerning selection of breast tissue-specific promoters, see, e.g., Patterson et al., 1999, Drugs Aging 14:75-90; Chung et al., 1999, Cancer Gene Ther. 6:99-106.

Examples of suitable promoters for driving human ovarian cancer-specific expression include the L-plastin promoter. See, e.g., Chung et al., 1999, Cancer Gene Ther 6:99-106). Regarding ovarian tumor selective expression, see, e.g., Tai et al., 1999, Cancer Res. 59:2121-2126.

When anti-metallothionein ribozymes are used in conjunction with radiation therapy according to the invention, additional specificity for cancer cells can be obtained by using a radiation inducible element, e.g., the early growth response 1 (EGR-1) promoter, p21 promoter, or tissue type plasminogen activator promoter, to drive ribozyme expression from a ribozyme-encoding gene therapy vector. For guidance concerning the use of radiation inducible elements in vectors useful in the invention, see, e.g., Marples et al., 2000, Gene Ther: 7:511-517. Types of radiation therapy that can be used in combination with anti-metallothionein ribozymes include external beam radiation for prostate cancer, brachytherapy for prostate cancer, ¹²⁵I for thyroid cancer, and ¹²⁵I estrogen for ovarian cancer.

In some embodiments of the invention, a ribozyme coding sequence and operably linked expression control sequences, including a suitable tissue-specific promoter, are incorporated into an adenoviral vector. For specific guidance concerning construction of adenoviral vectors containing a prostate tissue-specific promoter, see, e.g., Steiner et al., 2000, World J. Urol. 18:93-101; Greenberg et al., 1994, Mol. Endocrinol. 8:230-239; Steiner et al., 1999, Cancer Gene Ther. 6:456-464. For specific guidance concerning construction of adenoviral vectors containing an ovarian tissue-specific promoter, see, e.g., Chung et al., 1999, Cancer Gene Ther. 6:99-106.

Pharmaceutical Compositions

The ribozymes of the invention can be incorporated into pharmaceutical compositions. Such compositions typically include the ribozyme and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In some embodiments, a ribozyme of the invention is delivered directly to the desired site of action (e.g., a tumor) by injection. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished using nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Animal models for cancer (e.g., breast cancer, prostate cancer, and ovarian cancer) are known in the art. For example, see Steiner et al. (1999, Cancer Gene Therapy, 6:456-464) for a canine model useful for testing gene therapy methods related to prostate cancer. In addition, Greenberg et al., 1995, Proc. Natl. Acad. Sci., USA, 92:3439-43, describes a prostate cancer model in transgenic mice; Schroeder et al., 1976, Invest. Utol., 13:395-403, describes a nude mouse model of human prostatic adenoma and carcinoma; and Tekmal et al., 1996, Cancer Res., 56:3180-5, describes a breast cancer model in transgenic mice.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans more accurately. Levels in plasma may be measured, for example, by high performance liquid chromatography.

As defined herein, a therapeutically effective amount of a ribozyme (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The ribozyme can be administered one time per week for between about 1 to 10 weeks, for example, between 2 to 8 weeks, between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a ribozyme can include a single treatment or can include a series of treatments.

Exemplary doses include milligram or microgram amounts of the ribozyme per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a ribozyme depend upon the potency of the ribozyme with respect to its ability to affect metallothionein mRNA concentrations. When a ribozyme is to be administered to a mammal (e.g., a human) in order to modulate metallothionein expression, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The invention is further illustrated by the following examples. The examples are provided for illustrative purposes only, and are not to be construed as limiting the scope or content of the invention in any way.

### EXAMPLES

### Example 1

### Plasmids for Production of MT cRNAs

Two plasmids were constructed for the synthesis of MT-I and MT-II cRNA. MT-I and MT-II cDNAs, containing the entire coding sequences, were amplified by RT-PCR from hepatic total RNA samples using sequence specific primers (MT-I: forward primer 5′-GAA TTC CGT TGC TCC AGA TTC ACC AGA TC-3′ (SEQ ID NO: 8) and reverse primer 5′-GAA TTC TCA CAT GCT CGG TAG AAA ACG G-3′ (SEQ ID NO: 9); MT-II: forward primer 5′-TAG ATC TCC ACC TGC CGC CTC CA-3′ (SEQ ID NO: 10) and reverse primer 5′-TAG ATC TAC ACC ATT GTG AGG ACG CCC-3′ (SEQ ID NO: 11). A poly(A) tail was then added downstream of the two MT sequences.

These fragments were cloned individually into pBluescript SK+ vector (Stratagene, La Jolla, Calif.) to produce pBSMT-I, and pBSMT-II. The T3 promoter was used to in vitro transcribe the cRNA molecules by Maxiscript In Vitro Transcription kit (Ambion Inc., Austin, TX). Transcription from pBSMT-I and pBSMT-II produces native MT-I(polyA) and MT-II(polyA) cRNAs, respectively.

### Example 2

### MT-I and MT-II Ribozymes

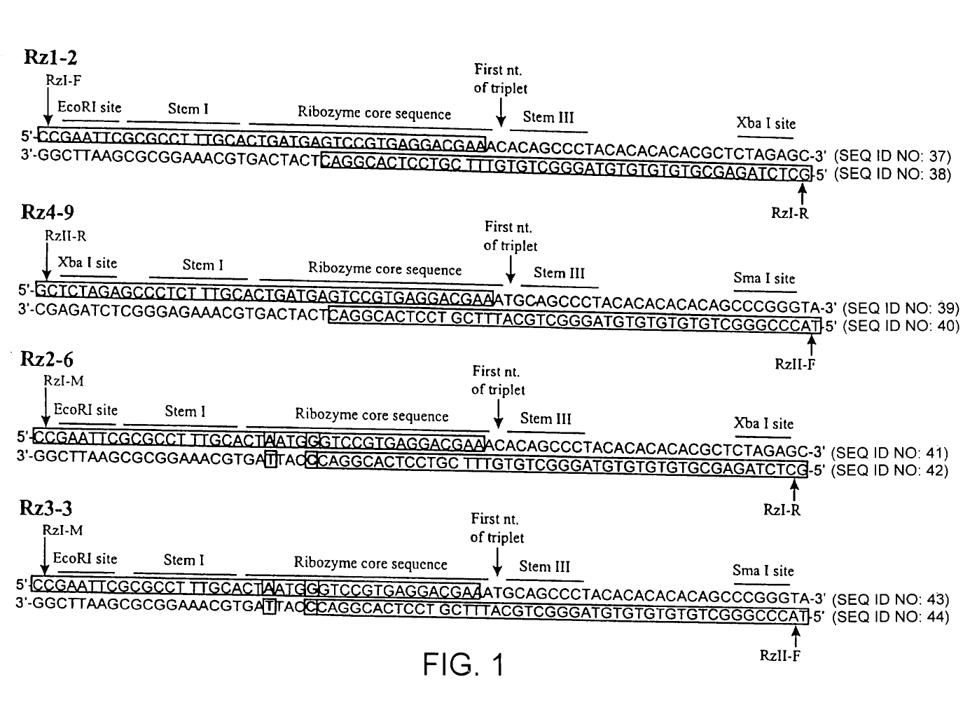

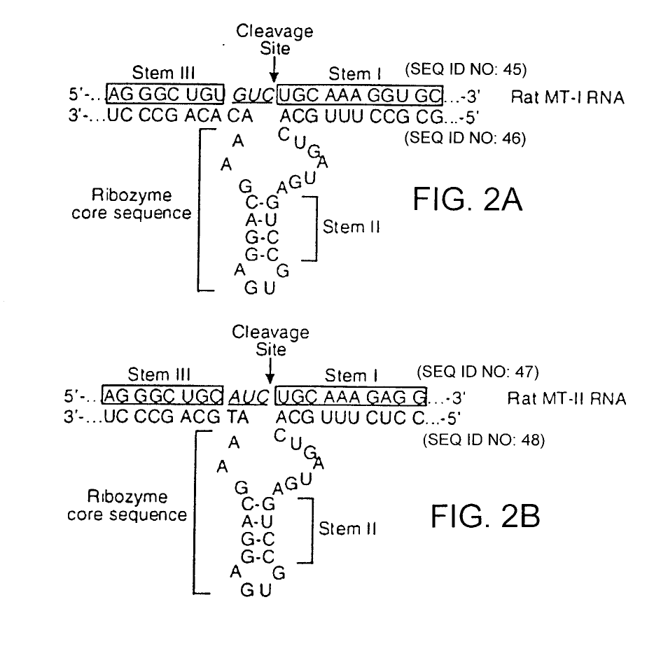

Computer assisted analyses were used to identify potential cleavage triplets in rat and mouse MT-I and MT-II mRNA sequences. First, computer analysis was used to search for potential cleavage triplets with the following characteristics: a 5′-NUH-3′ triple in which N is any nucleotide and H is A, U or C. It is known that G and C are the preferred first and third bases for hammerhead Rz cleavage triplets (Ruffner et al., 1990, Biochemistry 29:10695-10702; Shimayama et al., 1995, Biochemistry 34:3649-3654; Zoumadakis et al., 1995, Nucleic Acids. Res. 23:1192-1196). Secondary structure of target RNA was predicted using the MFOLD program (M. Zuker and P. Stiegler, Nucleic Acids Res., 9, 133-148 (1981); M. Zuker, The Use of Dynamic Programming Algorithms in RNA Secondary Structure Prediction in Mathematical Methods for DNA Sequences, M. S. Waterman ed. CRC Press, Inc., 159-184 (1989); and Mathews et al., Amer. Chem. Soc. Symp. Series, 682, 246-257, 1998) and the potential cleavage triplets that favor intermolecular hybridization were identified as described by James et al., 1997, Methods Mol. Biol. 74:17-26. Sequence analyses located a GUC triplet (+145-147) in the rat and mouse MT-I mRNA and an AUC site (+145-147) in MT-II transcripts of both species (FIGS. 1 and 2). Only one potential cleavage triplet was identified for each MT transcript, primarily due to the short length of the coding sequences. Both triplets were located in a region with little secondary structure. More importantly, they were in a highly conserved region of the MT mRNA molecule. This latter feature allowed was used to design ribozyme annealing arms (Stem I and Stem III) that could anneal to both rat and mouse MT mRNA (FIGS. 1-3).

To design annealing arms for the ribozymes, two factors were given particular consideration. Substrate specificity is related to the lengths of the base-pairing side arms flanking the cleavage triplet (annealing arms) (Herschlag, 1991, Proc. Natl. Acad. Sci. USA 88, 6921-6925; Beck et al., 1995, Nucleic Acids Res. 23:4954-4962; Lieber et al., 1995, Mol. Cell Biol. 15:540-551). However, long annealing arms may adversely affect enzyme activity by reducing ribozyme-substrate turnover rates. Based on these considerations, a 10-nucleotide (Stem I) and an 8-nucleotide (Stem II) annealing arm were designed for both ribozymes. The ribozyme core sequence (Stem II) (FIGS. 1 and 2) is primarily composed of conserved sequences and a four-nucleotide catalytic pocket. In the design of the mutant ribozymes, Rz2-6 and Rz3-3, corresponding to Rz1-2 and Rz4-9, respectively, two of the four nucleotides in the catalytic pocket were altered (see FIG. 1, the boxed nucleotides). These modifications were expected to abolish the catalytic activity in the ribozymes.

To construct the plasmids for ribozyme production, two pairs of overlapping oligonucleotides were purchased from Genosys (Woodlands, Tex.) (FIG. 1): Rz1-2 forward (RzI-F), 5′-CCG AAT TCG CGC CTT TGC ACT GAT GAG TCC GTG AGG ACG AA-3′ (SEQ ID NO: 12) and reverse (RzI-R), 5′-GCT CTA GAG CGT GTG TGT GTA GGG CTG TGT TTC GTC CTC ACG GAC-3′ (SEQ ID NO: 13); and Rz4-9 forward (RzII-F), 5′-TAC CCG GGC TGT GTG TGT GTA GGG CTG CAT TTC GTC CTC ACG GAC-3′ (SEQ ID NO: 14) and reverse (RzII-R), 5′-GCT CTA GAG CCC TCT TTG CAC TGA TGA GTC CGT GAG GAC GAA-3′ (SEQ ID NO: 15). PCR was performed to generate two extended double-stranded DNA products, Rz1-2 and Rz4-9, each containing their respective ribozyme sequence and two different terminal restriction sites for directional cloning (FIG. 1). A tandem ribozyme Rz(4-9/1-2) was constructed by fusing the two ribozymes, Rz(4-9) and Rz(1-2) via a segment of CA residues to separate the two catalytic units. Three expression vectors, pRz1-2, pRz4-9, and pRz(4-9/1-2) were then constructed by cloning the Rz1-2, Rz4-9, and Rz(4-9/1-2) into the pCLneo Mammalian Expression Vector (Promega, Madison, Wis.) which possesses a CMV promoter, a T3 promoter, a T7 promoter, and a polyA tail. Rzs were transcribed in vitro from the T3 or T7 promoters and used in cell-free studies. The vector pRz(4-9/1-2) was used in the cell transfection experiment permitting Rz(4-9/1-2) expression under the control of the CMV promoter.

A similar approach was used to generate plasmids for the production of enzymatically inactive ribozymes (FIG. 1). In these cases, an oligonucleotide with two altered nucleotides in the Rz core sequences was used to as the forward primer for the production of Rz2-6, a mutant of Rz1-2, or as the reverse primer in the synthesis of Rz3-3, a mutant of Rz4-9. The mutant oligonucleotide sequence, RzI-M, was as follows: 5′-CCG AAT TCG CGC CTT TGC ACT AAT GGG TCC GTG AGG ACG AA-3′ (SEQ ID NO: 16). The altered nucleotides boxed in FIG. 1. The PCR products were cloned into expression vectors and catalytically inactive Rzs, Rz2-6 and Rz3-3, representing mutants of Rz1-2 and Rz4-9 respectively, were synthesized via in vitro transcription.

### Example 3

### Rz1-2 and Rz4-9 Cleavage Activities

Bluescript plasmids containing the rat MT cDNA (pBSMT-I and pBSMT-2) were constructed as described above. A plasmid containing the mouse MT-I (pSP-MT-I) and a second plasmid (SDF 102.C1) containing MT-II cDNA (Durnam et al., 1980, Proc. Natl. Acad. Sci. USA 77:6511-6515) were linearized with HindIII and in vitro transcribed into labeled MT cRNAs with [α-32P]UTP, T3 RNA polymerase (Promega, Madison, Wis.), and the Maxiscript In Vitro Transcription Kit (Ambion, Inc, Austin, Tex.). Ribozymes were generated as unlabeled molecules from pRz1-2 and pRz4-9 using either the T3 or T7 promoter of the pClneo vectors, and unlabeled nucleoside triphosphates.

To test ribozyme activity, radiolabeled rat metallothionein cRNAs were synthesized by in vitro transcription from linearized plasmids and incubated with an excess of unlabeled ribozymes at 37° C. for 16 hours. The enzymatic activity was terminated with an equal volume of stop buffer. After denaturing at 95° C. for 2 minutes, the cleavage products were separated in a 6% polyacrylamide/8M urea gel, dried and exposed to X-ray film to obtain autoradiograms. The uncut rat MT-I (poly A) and MT-II (poly A) molecules were 435 nucleotides and 350 nucleotides, respectively. Rz1-2-mediated cleavage fragments of rat MT-I(poly A) were 360 nucleotides and 115 nucleotides in length. The cleavage fragments produced by Rz4-9 activity on MT-II(polyA) were 260 nucleotides and 90 nucleotides in length. Rz2-6 and Rz3-3 produced no cleavage products.

Initial experiments were designed to determine effective and linear catalytic temperature ranges for Rz1-2 and Rz4-9. A ribozyme and a labeled substrate were mixed in a 1:1 molar ratio in a 10 μL reaction mixture composed of 40 mM Tris-HCl (pH 7.5) and 20 mM MgCl₂ (previously determined to be optimal for cleavage activity). The reaction mixture was denatured for 2 minutes at 95° C. and then snapcooled on ice for 5 minutes. Catalytic reactions were conducted at 25° C., 37° C., and 50° C. for 1, 3, or 16 hours. A second series of experiments was performed to demonstrate sequence- and species-specificity. An excess of unlabeled anti-MT Rzs (Rz1-2 and Rz4-9) and their corresponding mutants (Rz2-6 and Rz3-3) were mixed with labeled rat or mouse MT-I or MT-II cRNA and incubated at 37° C. for 16 hours. At the end of these experiments, all catalytic reactions were terminated with an equal volume of stop buffer (10 mM EDTA, 90% formamide, 0.1% bromophenol blue, and 0.1% xylene cyanol). After denaturing at 95° C. for 2 minutes, the cleavage products were separated in a 6% polyacrylamide/8M urea gel, dried, and exposed to X-ray film to obtain autoradiograms. The sizes of the uncut rat MT-I (poly A) and MT-II (poly A) molecules were 435 nucleotides and 350 nucleotides, respectively. Rz1-2-mediated cleavage fragments of rat MT-I(poly A) were of 360 nucleotides and 115 nucleotides in length and those produced by Rz4-9 activity on MT-II(polyA) were 260 nucleotides and 90 nucleotides. Incubation with the mutant Rzs, Rz2-6 or Rz3-3, produced no products.

Ribozyme enzymatic kinetics on rat MT cRNAs or k_{cat}/Km values were analyzed under single turnover conditions according to the method of Heidenreich et al., 1992, J Biol. Chem. 267:1904-1909. Briefly, a metallothionein cRNA substrate (10 nM) was mixed with 1, 2, 4, 8, 16, 32, 64, or 128 nM of its sequence-complementary ribozyme in a 10 μl reaction buffer containing 40 mM Tris-HCl (pH 7.5) and 20 mM MgCl₂. Following a brief denaturing step at 95° C. for 2 minutes the enzymatic cleavage reaction was conducted at 37° C. for 1 hour. The cleavage products were separated by 6% polyacrylamide denaturing gel and autoradiographed at−70° C. The 5′and 3′ cleavage fragment band intensities for each RNA substrate were quantified by an image scanner, converted into digitized signals, quantified by the ImageQuant program (Molecular Dynamics, Inc., Sunnyvale, Calif.), and used to assess the percentage of cleavage. Percentage of cleavage was estimated as (intensity of the 5′ fragment+intensity of the 3′ fragment)/(intensity of the 5′ fragment +intensity of the 3′ fragment+intensity of the uncut substrate)×100. All experiments were performed in duplicate. K_{cat}/Km values as catalytic efficiency were obtained by plotting the intensity of the uncut substrate (S) against the ribozyme concentration ([Rz]) according to the following equation:

K=−1n S/t=[Rz]kcat/Km

$$k = - \frac{ln S}{t} = [ Rz ] \frac{kcat}{Km}$$

where k is the observed reaction rate and t is the reaction time of 1 hour.

### Example 4

### Mapping of MT-I and MT-II RNA Cleavage Sites

To verify that the ribozyme-induced cleavage on each metallothionein RNA occurred at the predicted sites (FIG. 2) primer extension analyses were conducted. Following the ribozyme-mediated cleavage of a metallothionein cRNA substrate, the cleavage products were subjected to primer extension in the presence of ³²P-UTP and a primer specific to the 3′-end of MT-I forward primer (see section a) or the MT-II forward primer. The reaction was started by the addition of 10 U SuperScript II RNasH reverse transcriptase and allowed incubate at 42° C. for 1 hour. The extended products were resolved directly in a 6% polyacrylamide denaturing gel with to a DNA sequencing ladder generated from cloned MT-I or MT-II cDNA using 5′-end radiolabeled primers (the same as those used in the primer extension studies) and the Thermo Sequenase Kit (Amersham, Arlington Heights, Ill.).

### Example 5

### Stable Transfectant Carrying Tandem Ribozymes

An immortalized, VP epithelial cell line, NbE-1 (Chang et al., 1989, Endocrinology 125:2719-2727),was routinely maintained in DMEM/F12 medium (Life Technologies, Gaithersburg, Md.) supplemented with 2 mM L-glutamine, 1 mM sodium pyruvate, 100 μM non-essential amino acids, 100 μg/ml penicillin, 100 μg/ml streptomycin, 50 μM μ-mercaptoethanol, ITS™ (Insulin-Transferrin-Selenium, Becton Dickson, Bedford, Mass.) and a 10% heat-inactivated, charcoal-stripped fetal bovine serum (csFBS, Sigma, St Louise, Mo.). Cells were plated at a density of 3×10⁵ cells per well in a 6-well plate. After 48 hours of culture cells were washed once with OPTI-MEM (Life Technologies, Gaithersburg, Md.) and transfected with 500 ng of pRz(4-9/1-2) using the Lipofectamine reagent (Life Technologies, Gaithersburg, Md.) according to the manufacturer's protocol. Transfectants were selected in 500 μg/ml G418 (Life Technologies, Gaithersburg, Md.) and after 3 weeks of exposure, individual clones were selected by ring-cloning, and expanded for further studies. NbE-1 clones that survived G418 selection were subjected to analyses of Rz(4-9/1-2) expression using semi-quantitative RT-PCR protocols (see below).

### Example 6

### Correlation of Ribozyme Expression and Metallothionein mRNA Expression

Expression of Rz (4-9/1-2) and MT-I and MT-II mRNA expression in stably transfected clones were quantified by semi-quantitative RT-PCR protocols. Total RNA was isolated by RNAzol B method (Tel-Test Inc., Friendswood, Tex.) with modifications described previously (Ghatak et al., 1996, Prostate 29:91-100). The quality of each total RNA sample was checked to ensure integrity and genomic DNA was eliminated by digestion with RNase-free DNase I (Sigma, St. Louis, Mo.). Total cellular RNA (1 μg) was reverse transcribed using the GeneAmp RNA PCR kit (Perkin-Elmer, Branchburg, N.J.) at 42° C. for 1 hour. Subsequently, 2 μl of the resulting reaction product was used for semi-quantification of cDNA of Rz(4-9/1-2), MT-I, MT-II and glyceraldehyde-3-phosphate-dehydrogenase (GADPH), a housekeeping gene. The PCR program was 1 minute at 94° C., 1 minute at 60° C. and 1 minute at 72° C. The PCR cycle numbers for each cDNA were optimized with multiple sets of preliminary experiments to ensure that amplifications of the products were linear with respect to the amount of input RNA. After PCR, the products were resolved on 2% agarose gel with ethidium bromide. The image was captured under UV trans-illumination with Type 665 negative film (Polaroid Co., Cambridge, Mass.) and intensity of the bands quantified by densitometric scanning followed by ImageQuant analyses (Molecular Dynamics, Sunnyvale, Calif.). Signal intensities of the targets [Rz(4-9/1-2), MT-I and MT-II] were normalized to those of the GAPDH PCR product to control for loading variations.

### Example 7

### Cytotoxicity Studies on Transfected NbE-1 Clones

Cells were plated in 96-well plates at a density of 5×10³ per well and maintained in DMEM/F12 (Life Technologies, Gaithersburg, Md.) with supplements. Twenty-four hours after plating, triplicate cultures were subjected to cadmium treatments for 4 days. CdCl₂ was added to the medium daily to achieve final concentrations of 3.1, 6.25, 12.5, 2.5, 50, 100, 200, or 400 μM. Following treatment, cell viability in cultures was determined by the MTT Cell Viability Assay Kit (Boehringer-Mannheim, Indianapolis, Ind.) in accordance to the vendor's instructions.

### Example 8

### Target Specificity and Cleavage Sites

Hammerhead ribozymes were tested for specific endonuclease activity under cell-free conditions. In vitro transcription from pBSMT-I and pBSMT-II using 2.5 μg HindIII linearized plasmid and T3 RNA polymerase yielded 5 μg of rat MT-I and MT-II cRNA. In parallel, mouse MT-I and MT-II cRNA were generated from plasmids containing mouse MT sequences (pSP-MT-I). These molecules containing the entire metallothionein coding region and a poly (A) tail 435 nucleotides (rat MT-I cRNA), 350 nucleotides (rat MT-II cRNA), 380 nucleotides (mouse MT-I cRNA) and 460 nucleotides (mouse MT-I cRNA) in length. They were used as substrate for the ribozyme reactions. Rz1 -2, the anti MT-I Rz, was found to be active on rat MT-I cRNAs but had no activity on rat MT-II cRNAs. Similarly, Rz4-9, the anti-MT-II ribozyme, cut only rat MT-II cRNAs and did not mediate rat MT-I cRNA cleavage. The ribozyme mutants, Rz2-6 and Rz3-3, had no catalytic activity. Similar sequence specificity data were obtained for mouse metallothionein cRNAs. All cleavage products were of the predicted sizes.

Primer extension analyses were used to confirm the cleavage sites in rat MT-I and MT-II cRNA (FIG. 2). The extension products of both ribozyme reactions co-migrate with an A, indicating that the phosphodiester bond cleavage was exactly 3′ to the GUC and AUC triplets in the MT-I and MT-II RNA substrates, respectively.

### Example 9

### Ribozyme Characteristics and Kinetics

Experiments were conducted to evaluate the constructed ribozymes. It was found that 20 mM of Mg²⁺ was optimal for both Rz1 -2 and Rz4-9 activity. Both ribozymes were active over a wide temperature range between 25-50° C. The ribozymes were most effective at 37° C. and remained active over a 16-hour period.

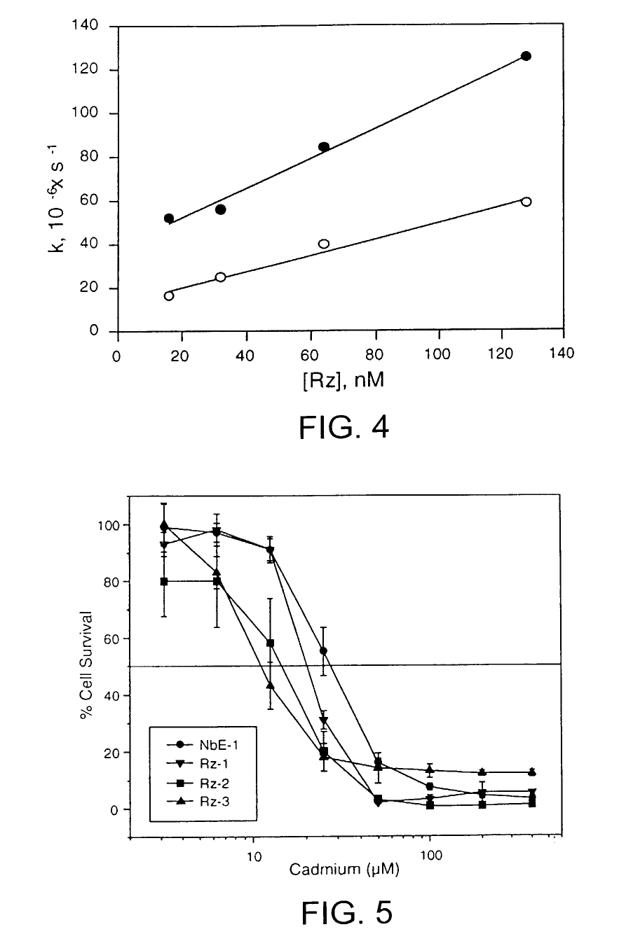

The catalytic efficiencies (k_{cat}/Km) of Rz1-2 and Rz4-9 on rat MT-I and MT-II cRNA were calculated from ribozyme reactions performed under single turnover conditions (Heidenreich et al., 1992, J Biol. Chem. 267:1904-1909). A constant amount of substrate (10 nM) and varying amounts (1-128 nM) of ribozyme was used (FIG. 4) and incubations were carried out at 37° C. for 1 hour. The relative catalytic efficiencies for Rz1-2 and Rz 4-9 678 M⁻¹s⁻¹ and 372 M⁻¹s⁻¹, respectively (FIG. 4).

These results demonstrate the suitability of ribozymes for use in the invention since they are relatively stable and are active over a range of temperatures.

### Example 10

### Transfection Studies and Cadmium Cytotoxicity Analyses

The immortalized rat VP epithelial cell line NbE-1 was transfected with the tandem ribozyme (Rz 4-9/Rz 1-2). Following G418 selection, three stably transfected, clonal lines, NbE-1 (Rz1), NbE-1 (Rz2) and NbE-1 (Rz3), were established. Using semi-quantitative RT-PCR protocols (Lee et al., 1999, Toxicol. Appl. Pharmacol. 154:20-27) it was established that two clones, NbE-1 (Rz2) and NbE-1 (Rz3), expressed sufficiently high levels of the tandem ribozyme construct to lower expression levels of MT-I and MT-II mRNA close to non-detectable levels while the NbE-1 (Rz1) line expressed low levels of the tandem ribozyme and relatively high levels of residue metallothionein messages when compared to parent NbE-1 cells (FIG. 5 insert). NbE-1 (Rz2) and NbE-1 (Rz3), but not NbE-1 (Rz1), were found to exhibit increased susceptibility to Cd-induced cytotoxicity (FIG. 5). The ID₅₀ of NbE-1 was around 30 μM, whereas values for NbE-1 (Rz2) and NbE-1 (Rz3) were approximately 10-15 μM.

Due to the short length of the coding sequence, only one potential cleavage triplet has been identified for each hammerhead ribozyme. A GUC triplet (+145-147) was identified in the MT-I mRNA and a AUC triplet (+145-147) found in the MT-II transcript. It was known that ribozymes display site preference on the triplet sequences in the following order: GUC, AUC>GUA, AUA, CUC>>GUU (Ruffner et al., supra; Shimayama et al., supra; Zoumadakis et al., supra; James et al., supra), with the GUC triplet yielding the highest rate of cleavage. In accordance with these reports, we observed a higher catalytic efficiency (678 M⁻¹s⁻¹) for Rz1-2 and a slightly lower efficiency for Rz4-9 (372 M⁻¹s⁻¹). In designing the annealing arms of the two ribozymes, we took into consideration that both turnover and substrate specificity can be affected by their lengths. It was demonstrated that the Rz1-2 and Rz4-9, each with 8-10 nucleotides in their annealing arms, were specific to their target substrates and exhibited catalytic efficiencies comparable to those displayed by ribozymes recognizing long substrates (˜10³ M⁻¹S⁻¹; Birikh et al., 1997, Eur. J Biochem. 245:1-16).

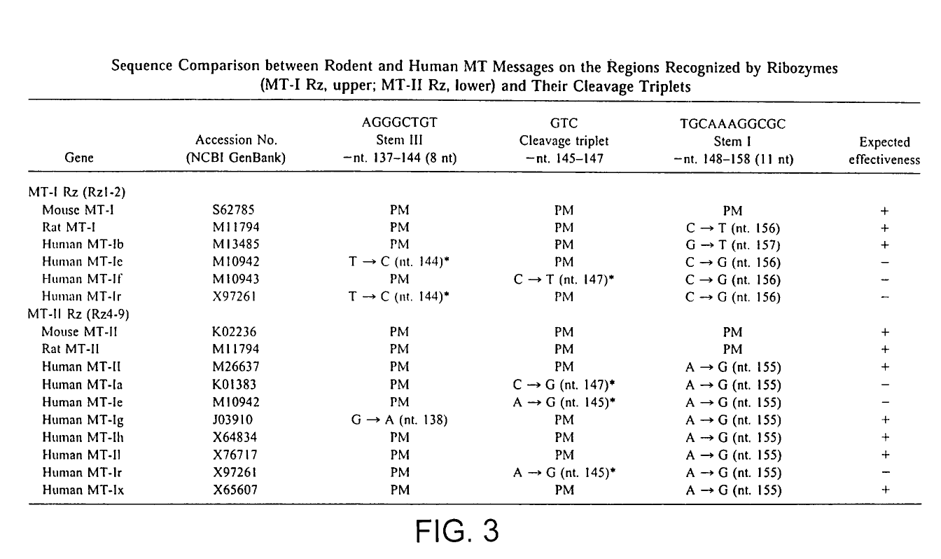

Sequence comparison between rodent and human metallothionein messages revealed that the rodent ribozymes may have cleavage activities on several human transcripts (FIG. 3). For example, Rz1-2 was predicted to cut human MT-Ib and Rz4-9 to cut human MT-II, MT-Ig, Ih, Il, and Ix. However, they were expected to have no activities on MT-Ia, Ie, If, Ir, due primarily to mismatched bases in either the side arms or the cleavage triplet (FIG. 3). These predictions were made based on the findings of Werner et al. 1995, Nucleic Acids Res. 23:2092-2096). A mismatch in the innermost base of Stem I was found to affect the chemical cleavage step, while more distal mismatches were shown to have no such effect. Moreover, mismatches in any of the four innermost base pairs of Stem III were found to exert deleterious effect on the cleavage reaction. Based on these observations on the effects of mismatches on ribozyme activity, only a small number of designed ribozymes would be needed to target all 17 metallothionein isoforms in human cells, because the targeted cleavage site is located in a highly conserved region of all metallothionein transcripts.

Expression of the tandem ribozyme construct, Rz(4-9/1-2), was shown to be effective in simultaneously down-regulating MT-I and MT-II mRNA in two stably transfected clones, NbE-1(Rz2) and NbE-1 (Rz3). More importantly, message down-regulation in these clones was found to be associated with an increase in cellular susceptibility to cadmium cytotoxicity.

### Example 11

### Ribozyme Effects in Human Cancer Cells

Transient Transfections

The effect of the constructed ribozymes in human cancer cells was examined. The human prostate cancer cell line, PC-3, and the ovarian cancer cell line, SKOV-3 cells, were purchased from the American Type Culture Collection (Manassas, Va.)(ATCC No. PC-3-CRL-1435, SKOV-3: HTB-77). For routine maintenance, the PC-3 cells were grown in DMEM/F-12 supplemented with steroid-depleted (charcoal stripped), heat-inactivated fetal bovine serum (FBS), 2 mM L-glutamine, 1 mM sodium pyruvate, 0.1 M non-essential amino acids, Penecillin/Streptomycin/Fungizone (P/S/F), 0.05 mM β-mercaptoethanol (Sigma Co), and 1% Insulin-Transferrin-Selenium mixture (ITS; BD Biosciences, Bedford, Mass.)+TM. The SKOV-3 cells were maintained in RPMI 1640 with heat-inactivated FBS supplemented with 2 mM L-glutamine, 1 mM sodium pyruvate, 0.1 M non-essential amino acids, P/S/F, 0.05 mM β-mercaptoethanol (Sigma Co), and 1% ITS+™. All cell cultures were incubated at 37° C. under a 5% CO₂ atmosphere.

The Rz 4-9, a ribozyme targeting rodent metallothionein-II was predicted to cleave human metallothionein-IIa mRNA. A sequence encoding Rz4-9 was cloned into the pCLneo Mammalian Expression Vector (Promega, Madison, Wis.) using a restriction site so that ribozyme expression would be driven by a CMV promoter and the transcript stabilized by a polyA tail. Two types of negative controls were used. One negative control was Rz3-3, a mutant ribozyme containing two altered nucleotides in the ribozyme core sequence targeting metallothionein-II. Rz 3-3 should bind to MT-II mRNA in a manner similar to Rz4-9, via its antisense arms, but was found to exhibit no enzymatic activity in vitro or in cellulo. The second negative control was the empty pCl-NEO vector (Promega, Wis.). The two ribozymes, Rz4-9 and Rz3-3 were cloned into this vector for the transient transfection studies.

To examine the efficacy of the constructed ribozyme (Rz4-9) in a cell, PC-3 and SKOV-3 cell lines were seeded at a density of 5×10³ each and grown in six well plates to sub-confluence (60-70%). Prior to transfection, the cells were washed and incubated for 1 hour in their respective supplemented media but without serum or antibiotics. Subsequently, 0.5, 1, and 2 μg/ml of the Rz4-9-encoding vector, and 2 μg/ml of the Rz3-3-encoding vector, 2 μg/ml of the pCL-NEO control vector were transfected into PC-3 and SKOV-3 cells, using Lipofectamine-PLUS (Life Technologies) according to vendor's instructions. The cells were incubated with the DNA for 3 hours at 37° C. to ensure uptake. At the end of 3 hours, heat-inactivated fetal bovine serum was added to bring the serum concentration to 10% for SKOV-3 and 5% for PC-3. After incubating for an additional 48 hours, the cells were washed and processed to determine the levels of MT-IIa mRNA using RT-PCR. Cell survival was determined by direct cell counting. Flow cytometry was used to determine the percentage of cells undergoing apoptosis. Parallel cultures were also processed for total RNA extraction and for cell cycle distribution analysis using fluorescence-activated cell sorting (FACS). For determining the efficiency of transfection, a pSV-β-galactosidase construct (Promega) was transfected using the procedure described above. Twenty-four hours following transfection, the β-galactosidase enzyme activity was assayed by the β-galactosidase reporter assay system (Promega) according to the manufacturer's instructions.

RNA Extraction and Semi-Quantitative RT-PCR Analyses

Because primary cultures of prostate epithelial cells, established from single biopsies and cell cultures were predicted to contain a limited number of cells and only small amounts of total RNA, semi-quantitative RT-PCR, was used since it requires only 1-2 mg total cellular RNA, to quantify and compare transcript levels. Semi-quantitative RT-PCR was compared to a competitive RT-PCR protocol, which permits mRNA to be quantified to absolute values. Comparable results were found with the two methods.

Total cellular RNA was isolated 24 hours after transfection using TRI reagent (Sigma, Mo.) according to protocols provided by the vendor. The quality of each total RNA sample was monitored and controlled by the following steps: 1) measurement of optical density, 2) running of a denaturing RNA gel capable of detecting possible RNA degradation, as judged by the integrity and intensity of the 18S and 28S ribosomal RNA signals detected by ethidium bromide, and 3) conducting semi-quantitative RT-PCR amplification of the 18S ribosomal RNA (18S rRNA) at low cycle numbers to ensure RNA integrity and quality.

Routinely, one mg of total cellular RNA was reverse-transcribed using the GeneAmp RNA PCR kit (Applied Biosystems, Foster City, Calif.) and 1-2 μl out of 50 μl of the resulting cDNA was used in each PCR. Intron-spanning primers targeting MT-IIa were designed using the Primer3 Output program, which is available at http://www.genome.wi.mit.edu/ cgi-bin/primer/primer3.cgi. The primers were designed to span the cleavage site (which is located at nucleotide 137-154 of human MT-IIa mRNA), so that PCR products would be generated only if there was un-cleaved MT-IIa mRNA were present. Primers designed to amplify from c-myc, bCI-2, MT-1F, or 18S rRNA cDNA, but not from their corresponding genomic DNA counterparts, were also selected from sequences generated by the Primer3 Output program.

All PCR conditions were optimized for quantification of relative message contents under non-saturating conditions. Hot-start PCR using AmpliTaq Gold™ DNA polymerase (Perkin-Elmer) was used in all the amplification reactions. The enzyme was activated by pre-heating the reaction mixtures at 95° C. for 8 minutes prior to PCR. Primer sequences and PCR conditions for MT-IIa and 18S ribosomal RNA were as follows. For MT-IIa cDNA amplification, forward primer was 5′-CAACCTGTCCCGACTCTAGCC-3′ (SEQ ID NO: 17) (nt 21-41) and the reverse primer was 5′-GGTCACGGTCAGGGTTGTAC-3′ (SEQ ID NO: 17) (nt 306-325). PCR was carried out under standard conditions with 30 cycles of denaturing (95° C. for 1 minute), annealing (55° C. for 1 minute) and extension (72° C. for 2 minutes), to amplify a 300 bp fragment corresponding to the MT-IIa mRNA. For amplification of the 18S ribosomal RNA, the forward primer was 5′-TGA GGC CAT GAT TAA GAG GG-3′ (SEQ ID NO: 19) as the sense primer and 5′-CGC TGA GCC AGT CAG TGTAG-3′ ( SEQ ID NO: 20) as the anti-sense primer to amplify a 623 bp 3′ fragment under PCR conditions similar to that of those used for MT-IIa cDNA amplification, except that it was carried out for 20 cycles at an annealing temperature of 60° C.

For experiments in which expression of bc1-2 and c-myc were examined, similar PCR conditions, with the exception of raising the annealing temperature to 60° C., were used to obtain a 294 bp amplimer from the bc1-2 cDNA and a 350 bp PCR product from c-myc cDNA.

For amplification of MT-1F from cDNA, the forward primer used was 5′-AGTCTCTCCTCGGCTTGC-3′ (nt 472-489; SEQ ID NO: 21) and the reverse primer was 5′-ACATCTGGGAGAAAGGTTGTC-3′ (nt 1603-1623; SEQ ID NO: 22). For amplification from bc1-2 cDNA, the forward primer was 5′-TGCACCTGACGCCCTTCAC-3′ (nt 386-404; SEQ ID NO: 23) and the reverse primer was 5′-AGACAGCCAGGAGAAATC-AAACAG-3′ (nt 655-679; SEQ ID NO: 24). For amplification from c-myc cDNA, 5′-CCACCACCAGCAGCGACTCTG-3′ (nt 1295-1315; SEQ ID NO: 25) was used as the sense primer and 5′-CCAAGACGTTGTGTGTTCGC-3′ (nt 1625-1645; SEQ ID NO: 26) as the antisense primer.

Extensive preliminary RT-PCRs were run for each transcript to optimize the conditions for quantification of relative message contents under non-saturating conditions and linearity between input RNA contents and signal intensities of the amplified product was always established for each transcript in multiple total RNA samples. Hot start PCR using AmpliTaq Gold™ DNA polymerase (Perkin-Elmer, CT) was employed in all amplification reactions to minimize non-specific product amplification. PCR product from each cDNA reverse transcribed from at least two different RNA samples were sub-cloned into a TA cloning vector (Invitrogen, CA) to verify the nucleotide sequence.

After amplification, PCR products were resolved by electrophoresis in 1.5% agarose gels containing ethidium bromide and images were captured under UV trans-illumination. The fluorescence images were captured on 665 negative film (Polaroid Co, Cambridge, Mass.), converted into digitized signals with an image scanner, and intensity of each band quantified by ImageQuant™ (Molecular Dynamics, Sunnyvale, Calif.) from the area under each peak. Signal intensities PCR products from MT-IIa were normalized to those from 18S ribosomal RNA cDNA to generate arbitrary units of relative abundance (ratios of the intensity of target PCR product to the intensity of 18S ribosomal RNA product). The reproducibility of the quantitative measurements was evaluated by three independent replicate cDNA synthesis and PCR runs.

Cell Cycle Analyses

For detection of cell viability, transient transfection of PC-3 or SKOV cells with plasmid DNA of Rz4-9 vector and empty vector were performed. PC-3 and SKOV-3 cell lines were seeded (5×10³ each) and grown in six well plates to sub-confluence. Ribozyme transfection was performed as described previously. At 18 hours post-transfection, the media containing the Lipofectamine-PLUS was replaced with normal growth medium containing serum, and the cells were allowed to grow for another 24 hours (total of 48 hours growth post-transfection). Subsequently, the medium was removed and the cells harvested for direct cell counting using trypan blue exclusion. The number of trypan blue-excluding cells was determined microscopically on hemacytometer. Six wells were counted for each test.

For detection of the hypodiploid cell population (cell cycle distribution) transfections were performed as described above. Forty-eight hours following transfection, the cells were fixed in ice-cold 70% ethanol for 24 hours at −20° C. The cells were subsequently pelleted and resuspended in buffer containing 9 parts 0.05 M Na₂HPO₄ 1 part 25 mM citric acid, and 0.1% Triton X-100 (pH 7.8) for 2 hours. The cells were subsequently pelleted and re-suspended in a buffer (10 mM PIPES, 0.1 N NaCl, 2 mM MgCl₂ and 0.1% Triton X-100) containing 20 mg/ml propidium iodide and 50 mg/ml RNAse (pH 6.8) and incubated in the dark at room temperature for 30 minutes. Cell cycle distribution and DNA content analysis were assayed according to Nusse et al. (1990, Cytometry 11:813-21) with a fluorescence-activated cell sorter, FACScan flow cytometer (Becton Dickinson, Mountain View, Calif.) and analyzed by the Cell Cycle Multi-Cycle system (Phoenix Flow System, San Diego, Calif.). Approximately 15,000 singlet events were collected and cell-cycle distribution was determined using Modfit LT Version 2 software (Verity House, Inc. Topsham, Me.).

Annexin V-FITC-PI Staining for Apoptotic Cells

Transient transfection of PC-3 or SKOV-3 cells with plasmid DNA of Rz4-9 vector, Rz3-3 vector, and an empty vector were performed as described above. Forty-eight hours following cell transfection, the culture medium was removed and the cells were washed in ice-cold phosphate buffered saline (pH 7.4). The washed cells were then stained with Annexin V-FITC and PI using the Annexin V-FITC apoptosis detection kit (Oncogene Research Products, Boston, Mass.) according to the manufacturer's instructions. Stained cells were analyzed using a by fluorescence activated cell sorting using a FACScan flow cytometer (Becton Dickinson, Mountain View, Calif.) and analyzed by Cell Cycle Multi-Cycle system (Phoenix Flow System, San Diego, Calif.).

Statistical Analyses

Statistical analyses were performed by using SYSTAT program (Course Technology, Inc., Cambridge, Mass.). Data were analyzed by one-way analysis of variance followed by the Tukey post-hoc comparison and a 95% confidence limit was used to determine significance. Relative mRNA abundance levels from four separate independent experiments were used to obtain averages.

Induction of Cell Loss in PC-3 and SKOV-3 Cancer Cells by Transient Transfection with the pCL-neo-Rz-4-9 Vector is Associated with a Reduction in Cellular MT-IIa, bcI-2, and c-myc mRNA Levels but not in Cellular MT-If Transcript Levels

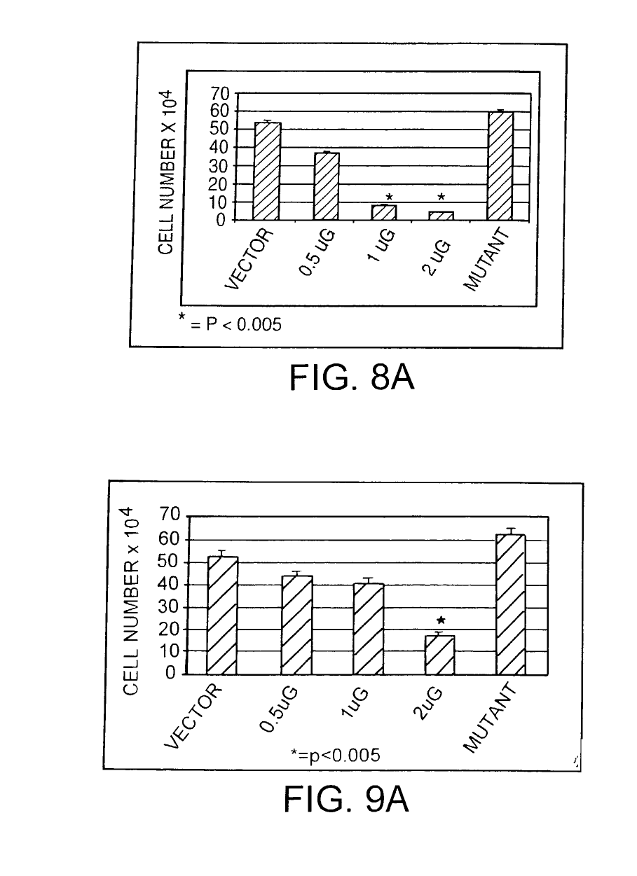

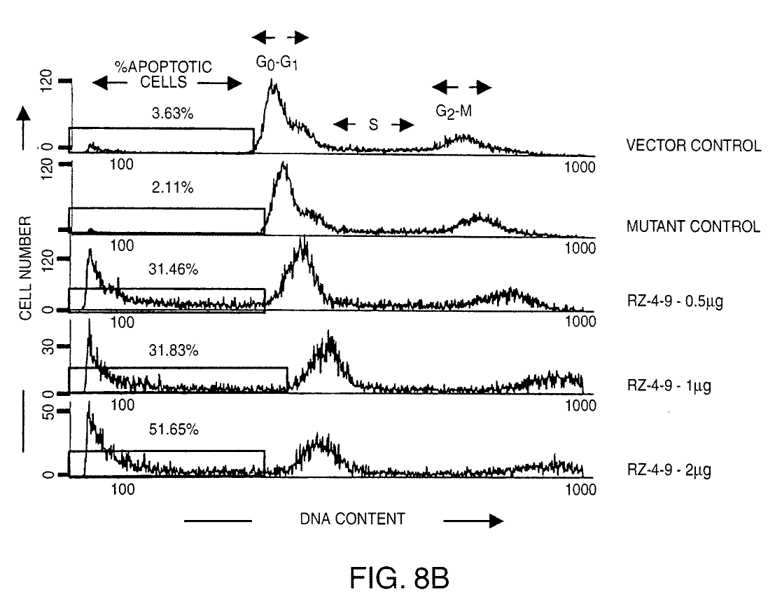

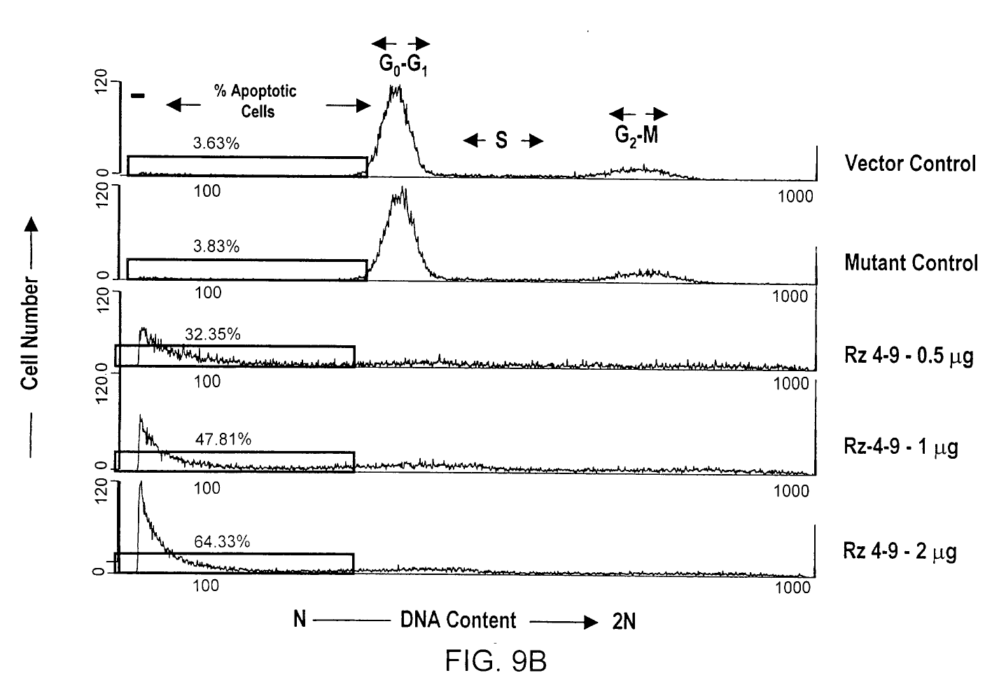

The ability of ribozymes targeted to MT-IIa to induce cell death was investigated. Forty-eight hours after transient transfection of cancer cells with pCL-neo-Rz4-9, pCl-neo-Rz3-3 or pCl-neo, cell numbers in cultures were determined by direct counting of both floating and adherent cells using the Trypan blue exclusion method. Cultures of PC-3 (FIG. 8A) and SKOV-3 cells (FIG. 9A) transfected with pCl-neo-Rz4-9 exhibited a marked, vector DNA concentration-dependent reduction in cell numbers. The number of viable PC-3 cells in transfected cultures was reduced by 33%, 85%, and 94%, respectively, when 0.5, 1 and 2 μg per well of a 6-well plate of pCl-neo-Rz-4-9 DNA was used. Similarly, SKOV-3 cell cultures transfected with 0.5, 1, and 2 μg of pCL-neo-Rz4-9 vector DNA exhibited a 17%, 23%, and 67% reduction in cell number, respectively. Cell cultures transfected with pCl-neo-Rz3-3 (the mutant Rz) or with the empty vector (controls) exhibited no reduction in cell number (FIGS. 8A and 9A). Transfection efficiencies were determined by using the pSV-β-galactosidase vector followed by the β-galactosidase reporter assay system and found to be 65% for PC-3 cells and 70% for SKOV-3 cells.

These data demonstrate that ribozymes targeted to metallothioneins can be used to induce cell death in cancer cells.

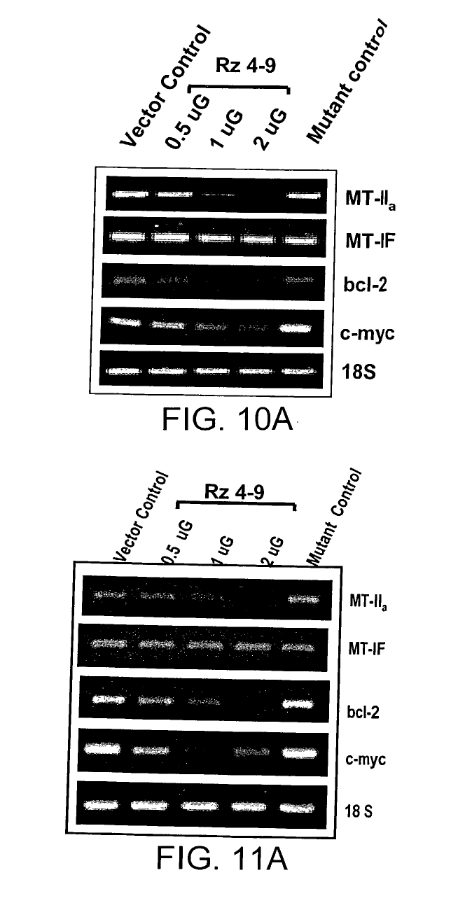

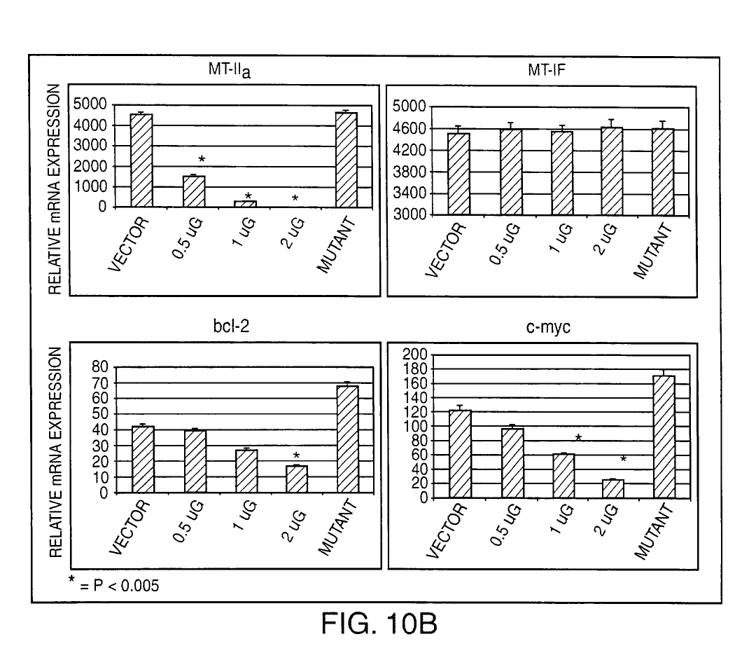

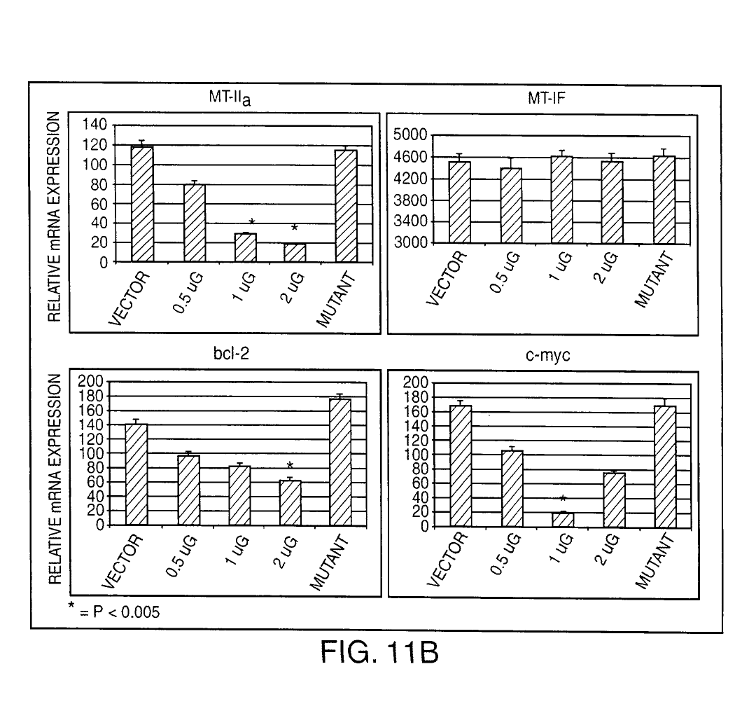

In parallel cultures, semi-quantitative RT-PCR was used to quantify the relative abundance of MT-IIa, MT-If, bcI-2, and c-myc mRNA in transfected cancer cell cultures. A vector DNA concentration-dependent reduction in MT-IIa mRNA was observed in total RNA samples isolated from PC-3 (FIG. 10A, see MT-IIa) and SKOV-3 (FIG. 11 A, see MT-IIa) cell cultures transfected with the pCl-neo-Rz4-9, but not in those transfected with the vector carrying the mutant Rz or the empty vector (FIG. 10A and 11A). A complete abolition of the MT-IIa message was achieved when 2 μg of pCl-neo-Rz4-9 DNA was used for cell transfection. However, treatment of PC-3 cells with 2 μg of either pCl-neo vector control or the mutant control (Rz 3-3) does not affect the expression of these transcripts. Concomitantly, bc1-2 and c-myc message levels were reduced in cell cultures transfected with the pCl-neo-Rz4-9 (FIGS. 10A and 11A, see bc1-2 and c-myc). No significant change in the MT-If transcript levels was observed (FIGS. 10A and 11A, see MT-If).

These data demonstrate that there is a concentration dependent down-regulation of MT-IIa, bc1-2, and c-myc in cancer cells treated with Rz-4-9. Thus, ribozymes targeting a specific metallothionein are effective for decreasing the amount of targeted mRNAs in cancer cells.

Rz-Induced MT-IIa Downregulation is Associated with Cell Loss Via Apoptosis

To determine whether cell loss observed in PC-3 and SKOV-3 cell cultures transfected with pCl-neo-Rz4-9 is attributable to apoptosis, cell cycle arrest, or a combination of the two, a FACS analysis on propidium iodide-stained cells obtained from transfected cell cultures was performed. Transfection of PC-3 cells (FIG. 8B) and SKOV-3 cells (FIG. 9B) with the MT-IIa targeting Rz, Rz4-9, induced a vector DNA dose-dependent increase in cells with hypodiploid DNA content 24 hours post-transfection. Previous studies have shown that cells presenting this feature in FACS analyses are those wherein DNA is fragmented at the internucleosomal linker regions and which then die by apoptosis (Fesus et al., 1991, Eur. J Cell Biol. 56:170 -177; Jacotot et al., 2000 Pathol Biol (Paris) 48:271-279). PC-3 cell cultures transfected with either the empty vector (FIGS. 8B and 9B, Vector Control) or the pCl-neo-Rz3-3 (FIGS. 8B and 9B, Mutant Control) have only 2-3% of cells with hypodiploid DNA contents. Transfection of PC-3 cells with 0.5, 1 and 2 μg pCl-neo-Rz4-9 resulted in a progressive increase in cells with hypodiploid DNA from 32% to 52%. Similarly, transfection of SKOV-3 cells with pCl-neo-Rz-4-9 also induced a vector DNA concentration-dependent increase in cells with hypodiploid DNA contents from 32% to 64%.

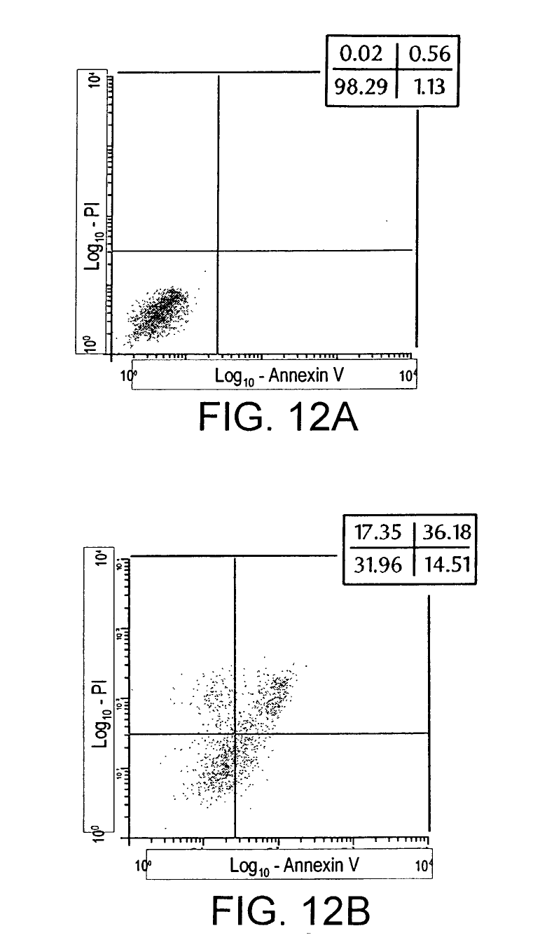

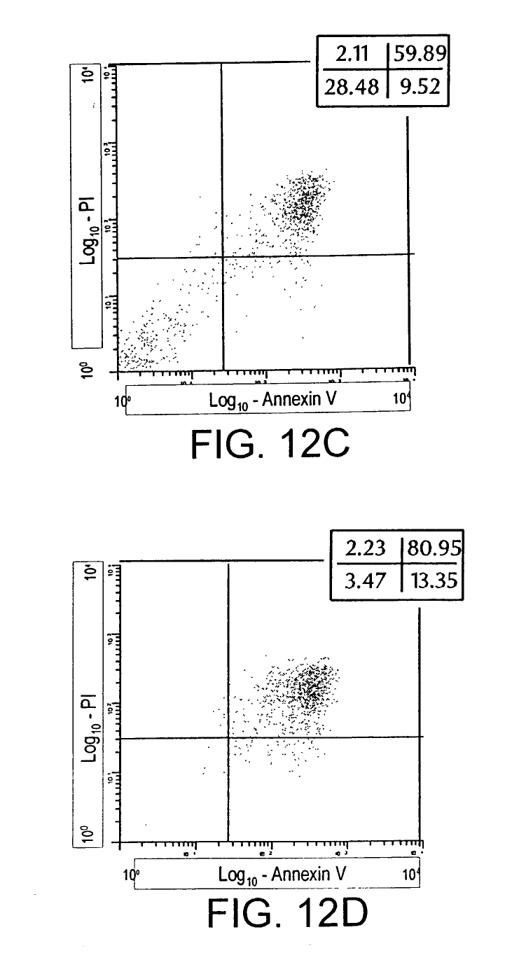

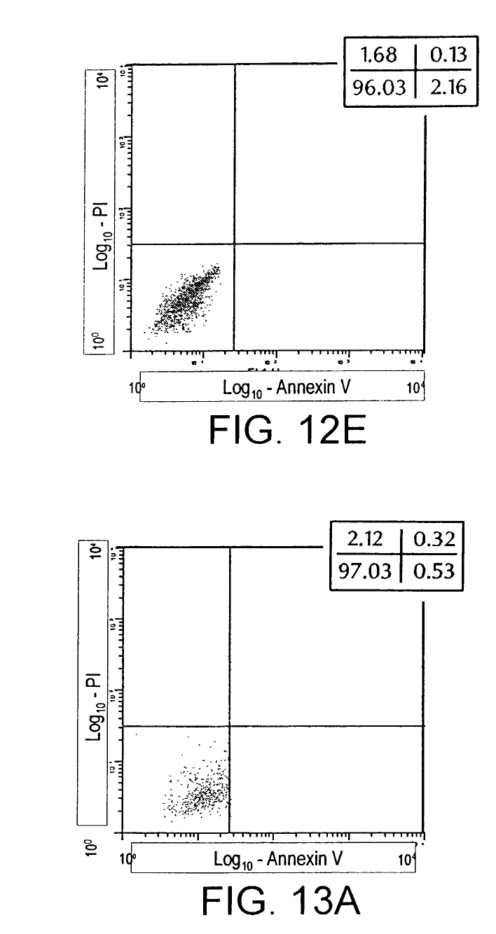

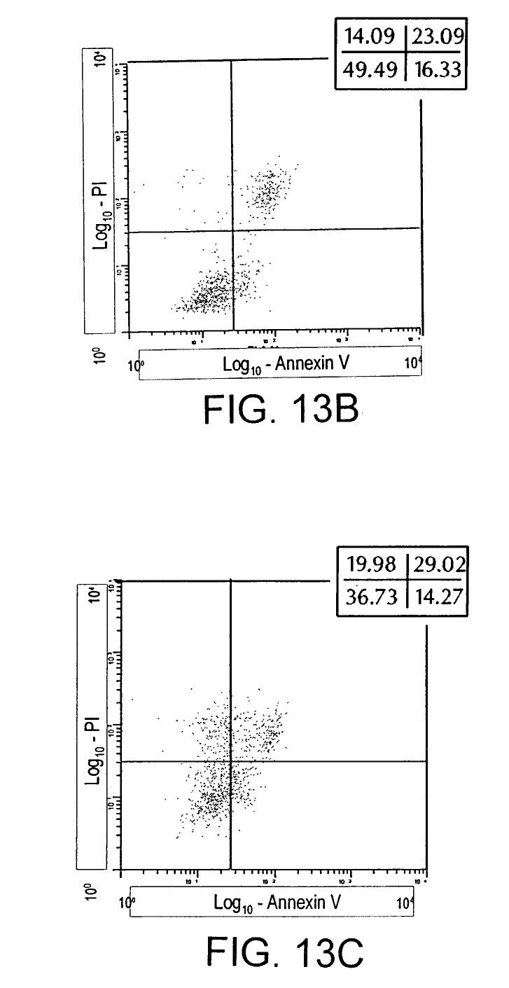

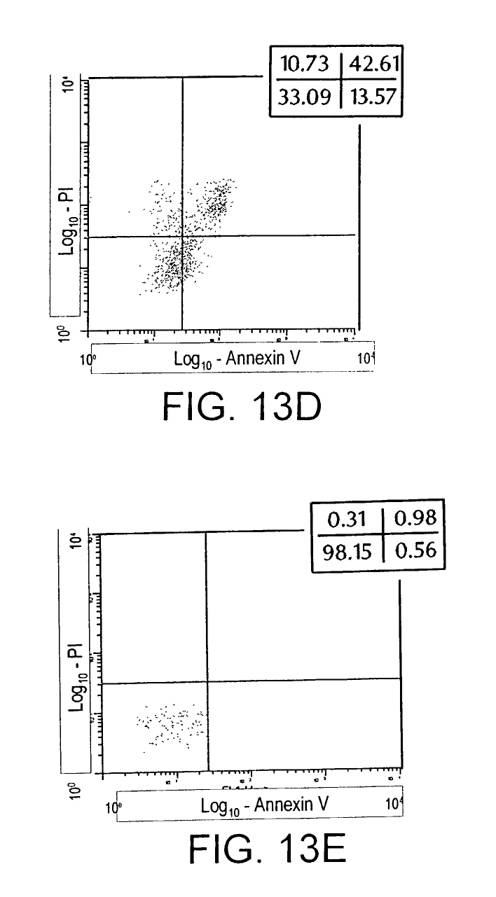

For confirmation of cell death via apoptosis, double stained transfected cells were double-stained with AnnexinV-FITC and PI to identify the early (Annexin V-positive and PI-negative) and late apoptotic (Annexin V-positive and PI-positive) populations in PC-3 and SKOV-3 cell cultures following transfection of various vectors. In PC-3 cell cultures, transfected with the empty pCl-neo vector or the pCl-neo-Rz-3-3 (mutant ribozyme) vector, only a very small number of late apoptotic cells were observed. AnnexinV- and PI-positive cells only comprised 0.56% (FIG. 12A) or 0.13% (FIG. 12B) of the total cell population, respectively in these cultures. In contrast, a vector DNA concentration-dependent increase in late apoptotic cell numbers was noted in PC-3 cells transfected with 0.5μg, 1 μg, or 2 μg of the pCl-neo-Rz-4-9 vector (FIGS. 12B, 12C, and 12D). Cells in late apoptotic phase reached 36.18%, 59.89%, or 80.95%, respectively, in these cultures. Similarly, following transfection of SKOV-3 cells with the pCl-neo vector or the pCl-neo-Rz-3-3 vector, only low numbers (0.32% or 0.98%, respectively) of late apoptotic cells were found in the cultures (FIGS. 13A and 13E). The results in SKOV-3 cells were similar to those obtained in PC-3 cells. A vector DNA concentration-dependent increase in late apoptotic cell numbers was observed in cultures transfected with 0.5μg, 1 μg, or 2 μg per well of a 6-well plate of pCl-neo-Rz-4-9 vector. Late apoptotic cell numbers comprised 23.09%, 29.02%, or 42.61% of the cell population, respectively, in the aforementioned cultures (FIGS. 13B, 13C, and 13D).

These data demonstrate that expression of Rz-4-9, a ribozyme designed to cleave MT-IIa, in PC-3 and SKOV-3 cells induced a ribozyme concentration-dependent diminution of human MT-IIa transcripts that is accompanied by dramatic cell death via apoptosis and down regulation of bc1-2 and c-myc expression. Expression of the ribozyme did not induce a reduction in the levels of a closely related transcript, MT-If mRNA, nor did it affect expression of housekeeping genes. These characteristics show that ribozymes targeted to a metallothionein are effective in inducing apoptotic cell death in cancer cells and is useful as a therapeutic tool for treating cancer.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of inducing apoptosis in a human cancer cell, the method comprising: introducing into the cell a ribozyme that inhibits metallothionein expression, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

2. The method of claim 1, wherein the human cancer cell is a cell in vitro.

3. The method of claim 1, wherein the human cancer cell is a cell in vivo.

4. The method of claim 3, wherein introducing the ribozyme into the cell comprises injecting the ribozyme directly into a tumor comprising the cell in vivo.

5. The method of claim 1, wherein the ribozyme is a hammerhead ribozyme.

6. The method of claim 1, wherein the human cancer cell is a prostate cancer cell, a breast cancer cell, or an ovarian cancer cell.

7. A method of inducing apoptosis in a human cancer cell, the method comprising:
introducing into the cell a nucleic acid vector comprising a nucleotide sequence encoding a ribozyme that inhibits metallothionein expression, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

8. The method of claim 7, wherein the human cancer cell is a cell in vitro.

9. The method of claim 7, wherein the human cancer cell is a cell in vivo.

10. The method of claim 7, wherein introducing the vector into the cell comprises injecting the vector directly into an in vivo tumor comprising the cell.

11. The method of claim 7, wherein the human cancer cell is a prostate cancer cell, a breast cancer cell, or an ovarian cancer cell.

12. The method of claim 11, wherein the nucleotide sequence is operatively linked to a prostate tissue-specific promoter, a breast-specific promoter, or an ovary-specific promoter.

13. A method of inhibiting growth of a tumor, the method comprising introducing into cells of the tumor a ribozyme that inhibits metallothionein expression, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

14. The method of claim 13, wherein introducing the ribozyme into the cells of the tumor comprises injecting the ribozyme directly into the tumor.

15. The method of claim 13, wherein the tumor is a human prostate tumor, human breast tumor, or human ovarian tumor.

16. A method of inhibiting growth of a tumor, the method comprising introducing into cells of the tumor a nucleic acid vector comprising a nucleotide sequence encoding a ribozyme that inhibits metallothionein expression, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

17. The method of claim 16, wherein introducing the vector into the cells of the tumor comprises injecting the vector directly into the tumor.

18. The method of claim 16, wherein the tumor is a human prostate tumor, a human breast tumor, or a human ovarian tumor.

19. The method of claim 16, wherein the nucleotide sequence is operatively linked to a prostate tissue-specific promoter, a breast tissue-specific promoter, or an ovarian tissue-specific promoter.

20. A method of enhancing the effectiveness of cancer therapy, the method comprising: introducing into cancer cells of a patient a ribozyme that inhibits metallothionein expression; and administering to the patient a therapeutically effective amount of a cancer therapy agent, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

21. The method of claim 20, wherein introducing the ribozyme into the cells comprises injecting the ribozyme directly into an in vivo tumor comprising the cells.

22. The method of claim 20, wherein the tumor is a human prostate tumor, a human breast tumor, or a human ovarian tumor.

23. The method of claim 20, wherein the cancer therapy is chemotherapy.

24. The method of claim 23, wherein the cancer therapy agent is selected from the group consisting of cisplatin, estramustine, vinblastine, etopside, paclitaxel, taxotere, docetaxel, doxorubicin, ketocanazole, and cyclophosphamide.

25. The method of claim 20, wherein the therapy is radiation therapy

26. The method of claim 25, wherein the type of radiation therapy is selected from the group consisting of external beam radiation therapy, brachytherapy, and ¹²⁵I administration.

27. A method of enhancing the effectiveness of cancer therapy, the method comprising: introducing into the cancer cells of a patient a nucleic acid vector comprising a nucleotide sequence encoding a ribozyme that inhibits metallothionein expression; and administering to the patient a therapeutically effective amount of the cancer therapy, wherein the ribozyme comprises a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

28. The method of claim 27, wherein introducing the vector into the cells comprisesinjecting the vector directly into an in vivo tumor comprising the cells.

29. The method of claim 27, wherein the tumor is a human prostate tumor, a human breast tumor, or a human ovarian tumor.

30. The method of claim 27, wherein the type of therapy is radiation therapy.

31. The method of claim 30, wherein the type of radiation therapy is selected from the group consisting of external beam radiation therapy, brachytherapy, and ¹²⁵I administration.

32. The method of claim 27, wherein the type of therapy is chemotherapy.

33. The method of claim 32, wherein the cancer therapy agent is selected from the group consisting of cisplatin, estramustine, vinblastine, etopside, paclitaxel, taxotere, docetaxel, doxorubicin, ketocanazole, and cyclophosphamide.

34. A ribozyme comprising a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |

35. A vector comprising a nucleic acid molecule that encodes the ribozyme of claim 34.

36. The ribozyme of claim 34, wherein the nucleotide sequence is the sequence of Hu MT-Ia Rz, Hu MT-Ie/r Rz, or Hu MT-If Rz.

37. The ribozyme of claim 34, wherein the nucleotide sequence is the sequence of Hu MT-Ib Rz or Hu MT-Ighlx/-II Rz.

38. A vector comprising two or more ribozymes, each having a nucleotide sequence selected from the group consisting of:

| 5′-GCACUUCUCUCUGAUGAGUCCGUGAGGACGAAAU | (SEQ ID NO:1) |
|---|---|
| GCCCCUUUGC-3′ (Hu MT-Ia Rz); | |
| 5′-CCCCUUUGCACUGAUGAGUCCGUGAGGACGAAAC | (SEQ ID NO:2) |
| GCAGCCCU-3′ (Hu MT-Ie/r Rz); | |
| 5′-GCACUUCUCUCUGAUGAGUCCGUGAGGACGAAAC | (SEQ ID NO:3) |
| GCCCCUUUGC-3′ (Hu MT-If Rz); | |
| 5′-GAGCCUUUGCACUGAUGAGUCCGUGAGGACGAAA | (SEQ ID NO:4) |
| CACAGCCCU-3′ (Hu MT-Ib Rz); | |
| 5′-CCCCUUUGCACUGAUGAGUCCGUGAGGACGAAAU | (SEQ ID NO:5) |
| GCAGCCCU-3′ (Hu MT-Ighlx/-II Rz); | |
| 5′-GCGCCUUUGCACUGAUGAGUCCGUGAGGACGAAA | (SEQ ID NO:6) |
| CACAGCCCU-3′ (Rz1-2); and | |
| 5′-CCUCUUUGCACUGAUGAGUCCGUGAGGACGAAAU | (SEQ ID NO:7) |
| GCAGCCCU-3′ (Rz4-9). | |


SEQUENCE LISTING (68 sequences)

SEQ ID NO: 1 - RNA, 44 nt, Artificial Sequence
  Human MT-Ia Rz
gcacuucucucugaugaguccgugaggacgaaaugccccuuugc

SEQ ID NO: 2 - RNA, 42 nt, Artificial Sequence
  Human MT-Ie/r Rz
ccccuuugcacugaugaguccgugaggacgaaacgcagcccu

SEQ ID NO: 3 - RNA, 44 nt, Artificial Sequence
  Human MT-If Rz
gcacuucucucugaugaguccgugaggacgaaacgccccuuugc

SEQ ID NO: 4 - RNA, 43 nt, Artificial Sequence
  Human MT-Ib Rz
gagccuuugcacugaugaguccgugaggacgaaacacagcccu

SEQ ID NO: 5 - RNA, 42 nt, Artificial Sequence
  Human MT-Igh1x/-II Rz
ccccuuugcacugaugaguccgugaggacgaaaugcagcccu

SEQ ID NO: 6 - RNA, 43 nt, Artificial Sequence
  Rz1-2
gcgccuuugcacugaugaguccgugaggacgaaacacagcccu

SEQ ID NO: 7 - RNA, 42 nt, Artificial Sequence
  Rz4-9
ccucuuugcacugaugaguccgugaggacgaaaugcagcccu

SEQ ID NO: 8 - DNA, 29 bp, Artificial Sequence
  MT-I forward primer
gaattccgttgctccagattcaccagatc

SEQ ID NO: 9 - DNA, 28 bp, Artificial Sequence
  MT-I reverse primer
gaattctcacatgctcggtagaaaacgg

SEQ ID NO: 10 - DNA, 23 bp, Artificial Sequence
  MT-II forward primer
tagatctccacctgccgcctcca

SEQ ID NO: 11 - DNA, 27 bp, Artificial Sequence
  MT-II reverse primer
tagatctacaccattgtgaggacgccc

SEQ ID NO: 12 - DNA, 41 bp, Artificial Sequence
  Rz1-2 forward (RzI-F) primer
ccgaattcgcgcctttgcactgatgagtccgtgaggacgaa

SEQ ID NO: 13 - DNA, 45 bp, Artificial Sequence
  reverse primer (RzI-R)
gctctagagcgtgtgtgtgtagggctgtgtttcgtcctcacggac

SEQ ID NO: 14 - DNA, 45 bp, Artificial Sequence
  Rz4-9 forward primer (RzII-F)
tacccgggctgtgtgtgtgtagggctgcatttcgtcctcacggac

SEQ ID NO: 15 - DNA, 42 bp, Artificial Sequence
  Rz4-9 reverse primer (RzII-R)
gctctagagccctctttgcactgatgagtccgtgaggacgaa

SEQ ID NO: 16 - DNA, 41 bp, Artificial Sequence
  mutant oligonucleotide sequence, RzI-M
ccgaattcgcgcctttgcactaatgggtccgtgaggacgaa

SEQ ID NO: 17 - DNA, 21 bp, Artificial Sequence
  nt 21-41; for MT-IIa cDNA amplification, forward primer
caacctgtcccgactctagcc

SEQ ID NO: 18 - DNA, 20 bp, Artificial Sequence
  nt 306-325; for MT-IIa cDNA amplification, reverse primer
ggtcacggtcagggttgtac

SEQ ID NO: 19 - DNA, 20 bp, Artificial Sequence
  forward (sense) primer for amplification of the 18S ribosomal RNA
tgaggccatgattaagaggg

SEQ ID NO: 20 - DNA, 20 bp, Artificial Sequence
  anti-sense primer for amplificaiton of the 18S ribosomal RNA
cgctgagccagtcagtgtag

SEQ ID NO: 21 - DNA, 18 bp, Artificial Sequence
  nt 472-489; forward primer for amplification of MT-1F from cDNA
agtctctcctcggcttgc

SEQ ID NO: 22 - DNA, 21 bp, Artificial Sequence
  nt 1603-1623; reverse primer for amplification of MT-1F from cDNA
acatctgggagaaaggttgtc

SEQ ID NO: 23 - DNA, 19 bp, Artificial Sequence
  nt 386-404; forward primer for amplification from bcI-2 cDNA
tgcacctgacgcccttcac

SEQ ID NO: 24 - DNA, 24 bp, Artificial Sequence
  nt 655-679; reverse primer for amplification from bcI-2 cDNA
agacagccaggagaaatcaaacag

SEQ ID NO: 25 - DNA, 21 bp, Artificial Sequence
  nt 1295-1315; sense primer for amplification from c-myc cDNA
ccaccaccagcagcgactctg

SEQ ID NO: 26 - DNA, 20 bp, Artificial Sequence
  nt 1625-1645; anti-sense primer for amplification from c-myc cDNA
ccaagacgttgtgtgttcgc

SEQ ID NO: 27 - DNA, 40 bp, Artificial Sequence
  Human MT-Ia Rz forward primer
ccgaattcgcacttctctctgatgagtccgtgaggacgaa

SEQ ID NO: 28 - DNA, 45 bp, Artificial Sequence
  Human MT-Ia Rz reverse primer
gctctagagcgtgtgtgtgcaaaggggcatttcgtcctcacggac

SEQ ID NO: 29 - DNA, 40 bp, Artificial Sequence; identical to SEQ ID NO: 27
  Human MT-If Rz forward primer

SEQ ID NO: 30 - DNA, 45 bp, Artificial Sequence
  Human MT-If Rz reverse primer
gctctagagcgtgtgtgtgcaaaggggcgtttcgtcctcacggac

SEQ ID NO: 31 - DNA, 44 bp, Artificial Sequence
  Human MT-Ie/r Rz forward primer
tacccgggctgtgtgtgtgtagggctgcgtttcgtctcacggac

SEQ ID NO: 32 - DNA, 42 bp, Artificial Sequence
  Human MT-Ie/r Rz reverse primer
gctctagagccccctttgcactgatgagtccgtgaggacgaa

SEQ ID NO: 33 - DNA, 42 bp, Artificial Sequence; identical to SEQ ID NO: 32
  Human MT-Ighlx/-II Rz forward primer

SEQ ID NO: 34 - DNA, 45 bp, Artificial Sequence; identical to SEQ ID NO: 14
  Human MT-Ighlx/-II Rz reverse primer

SEQ ID NO: 35 - DNA, 41 bp, Artificial Sequence
  Human MT-Ib Rz forward primer
ccgaattcgagcctttgcactgatgagtccgtgaggacgaa

SEQ ID NO: 36 - DNA, 45 bp, Artificial Sequence; identical to SEQ ID NO: 13
  Human MT-Ib Rz reverse primer

SEQ ID NO: 37 - DNA, 71 bp, Artificial Sequence
  Rz1-2, RzI-F forward primer
ccgaattcgcgcctttgcactgatgagtccgtgaggacgaaacacagccctacacacacacgctctagagc

SEQ ID NO: 38 - DNA, 71 bp, Artificial Sequence
  Rz1-2, RzI-R reverse primer
gctctagagcgtgtgtgtgtagggctgtgtttcgtcctcacggactcatcagtgcaaaggcgcgaattcgg

SEQ ID NO: 39 - DNA, 72 bp, Artificial Sequence
  Rz4-9, RzII-R forward primer
gctctagagccctctttgcactgatgagtccgtgaggacgaaatgcagccctacacacacacagcccgggta

SEQ ID NO: 40 - DNA, 72 bp, Artificial Sequence
  Rz4-9, RzII-F reverse primer
tacccgggctgtgtgtgtgtagggctgcatttcgtcctcacggactcatcagtgcaaagagggctctagagc

SEQ ID NO: 41 - DNA, 71 bp, Artificial Sequence
  Rz2-6, RzI-M forward primer
ccgaattcgcgcctttgcactaatgggtccgtgaggacgaaacacagccctacacacacacgctctagagc

SEQ ID NO: 42 - DNA, 71 bp, Artificial Sequence
  Rz2-6, RzI-R reverse primer
gctctagagcgtgtgtgtgtagggctgtgtttcgtcctcacggacccattagtgcaaaggcgcgaattcgg

SEQ ID NO: 43 - DNA, 71 bp, Artificial Sequence
  Rz3-3, RzI-M forward primer
ccgaattcgcgcctttgcactaatgggtccgtgaggacgaaatgcagccctacacacacacagcccgggta

SEQ ID NO: 44 - DNA, 71 bp, Artificial Sequence
  Rz3-3, RzI-F reverse primer
tacccgggctgtgtgtgtgtagggctgcatttcgtcctcacggacccattagtgcaaaggcgcgaattcgg

SEQ ID NO: 45 - RNA, 22 nt, Artificial Sequence
  Rat MTpI RNA
agggcugugucugcaaaggugc

SEQ ID NO: 46 - RNA, 43 nt, Artificial Sequence; identical to SEQ ID NO: 6
  Rat MT-I RNA

SEQ ID NO: 47 - RNA, 21 nt, Artificial Sequence
  Rat MT-II RNA
agggcugcaucugcaaagagg

SEQ ID NO: 48 - DNA, 42 bp, Artificial Sequence
  Rat MT-II RNA
ccucuuugcacugaugaguccgugaggacgaaatgcagcccu

SEQ ID NO: 49 - DNA, 70 bp, Artificial Sequence
  Human MT-Ia Rz; RzI-F
ccgaattcgcacttctctctgatgagtccgtgaggacgaaatgcccctttgcacacacacgctctagagc

SEQ ID NO: 50 - DNA, 70 bp, Artificial Sequence
  Human MT-Ia Rz;  RzI-R, reverse primer
gctctagagcgtgtgtgtgcaaaggggcatttcgtcctcacggactcatcagagagaagtgcgaattcgg

SEQ ID NO: 51 - DNA, 72 bp, Artificial Sequence
  Human MT-Ie/r Rz; RzII-R, forward primer
gctctagagccccctttgcactgatgagtccgtgaggacgaaacgcagccctacacacacacagcccgggta

SEQ ID NO: 52 - DNA, 72 bp, Artificial Sequence
  Human MT-Ie/r Rz; RzII-F, reverse primer
tacccgggctgtgtgtgtgtagggctgcgtttcgtcctcacggactcatcagtgcaaagggggctctagagc

SEQ ID NO: 53 - DNA, 70 bp, Artificial Sequence
  Human MT-If Rz; RzI-F forward primer
ccgaattcgcacttctctctgatgagtccgtgaggacgaaacgcccctttgcacacacacgctctagagc

SEQ ID NO: 54 - DNA, 70 bp, Artificial Sequence
  Human MT-If Rz; RzI-R reverse primer
gctctagagcgtgtgtgtgcaaaggggcgtttcgtcctcacggactcatcagagagaagtgcgaattcgg

SEQ ID NO: 55 - DNA, 71 bp, Artificial Sequence
  Human MT-Ib Rz; RzI-F forward primer
ccgaattcgagcctttgcactgatgagtccgtgaggacgaaacacagccctacacacacacgctctagagc

SEQ ID NO: 56 - DNA, 71 bp, Artificial Sequence
  Human MT-Ib Rz; RzI-R reverse primer
gctctagagcgtgtgtgtgtagggctgtgtttcgtcctcacggactcatcagtgcaaaggctcgaattcgg

SEQ ID NO: 57 - DNA, 72 bp, Artificial Sequence
  Human MT-Ighlx/-II Rz; RzII-R forward primer
gctctagagccccctttgcactgatgagtccgtgaggacgaaatgcagccctacacacacacagcccgggta

SEQ ID NO: 58 - DNA, 72 bp, Artificial Sequence
  Human MT-Ighlx/-II Rz; RzII-F reverse primer
tacccgggctgtgtgtgtgtagggctgcatttcgtcctcacggactcatcagtgcaaagggggctctagagc

SEQ ID NO: 59 - RNA, 23 nt, Artificial Sequence
  Human MT-Ia RNA
gcaaaggggcaucagagaagugc

SEQ ID NO: 60 - RNA, 44 nt, Artificial Sequence; identical to SEQ ID NO: 1
  Human MT-Ia RNA Rz

SEQ ID NO: 61 - RNA, 22 nt, Artificial Sequence
  Human MT-Ib RNA
agggcugugucugcaaaggcuc

SEQ ID NO: 62 - RNA, 43 nt, Artificial Sequence; identical to SEQ ID NO: 4
  Human MT-Ib RNA Rz

SEQ ID NO: 63 - RNA, 21 nt, Artificial Sequence
  Human MT-I/ghlx-II RNA
agggcugcaucugcaaagggg

SEQ ID NO: 64 - DNA, 42 bp, Artificial Sequence
  Human MT-I/ghlx-II RNA Rz
ccccuuugcacugaugaguccgugaggacgaaatgcagcccu

SEQ ID NO: 65 - RNA, 23 nt, Artificial Sequence
  Human MT-If RNA
gcaaaggggcgucagagaagugc

SEQ ID NO: 66 - RNA, 44 nt, Artificial Sequence; identical to SEQ ID NO: 3
  Human MT-If RNA Rz

SEQ ID NO: 67 - RNA, 21 nt, Artificial Sequence
  Human MT-Ie/r RNA
agggcugcgucugcaaagggg

SEQ ID NO: 68 - RNA, 42 nt, Artificial Sequence; identical to SEQ ID NO: 2
  Human MT-Ie/r RNA Rz